(19) United States
(12) Patent Application Publication
(10) Pub. No.: US 2013/0101512 A1
(43) Pub. Date: Apr. 25, 2013

(54) CROSSLINKED POLYNUCLEOTIDE STRUCTURE

(71) Applicants: Chad A. Mirkin, Wilmette, IL (US); C. Shad Thaxton, Chicago, IL (US); David A. Giljohann, Chicago, IL (US); Joshua I. Cutler, Evanston, IL (US)

(72) Inventors: Chad A. Mirkin, Wilmette, IL (US); C. Shad Thaxton, Chicago, IL (US); David A. Giljohann, Chicago, IL (US); Joshua I. Cutler, Evanston, IL (US)

(21) Appl. No.: 13/634,434

(22) PCT Filed: Mar. 14, 2011
(86) PCT No.: PCT/US11/28380

§ 371 Date: Nov. 20, 2012

Related U.S. Application Data
(60) Provisional application No. 61/340,128, filed on Mar. 12, 2010.

(51) Int. Cl. C12Q 1/68; A61K 49/00

(52) U.S. Cl. CPC C12Q 1/6841; A61K 49/00
USPC 424/9.1; 506/16; 506/9

(57) ABSTRACT

The present invention provides structures formed from crosslinked polynucleotides, where a subset of the polynucleotides binds to a target under physiological conditions, where the signal group detectably changes upon binding.

### BACKGROUND

The natural defenses of biological systems for exogenous oligonucleotides, such as synthetic antisense DNA and siRNA, present many challenges for the delivery of nucleic acids in an efficient, non-toxic and non-immunogenic fashion. Indeed, because nucleic acids are negatively charged and prone to enzymatic degradation, researchers have historically relied on transfection agents such as cationic polymers, modified viruses, and liposomes to facilitate cellular entry and protect DNA from degradation. However, each of these materials is subject to several drawbacks, which include toxicity at high concentrations, inability to be degraded biologically, and severe immunogenicity.

Polyvalent nucleic acid-nanoparticle conjugates (inorganic nanoparticles densely coated with highly oriented oligonucleotides) pose one possible solution of circumventing these problems in the context of both antisense and RNAi pathways (N. L. Rosi et al., Science 312, 1027 (May 19, 2006); D. A. Giljohann et al., J. Am. Chem. Soc. 131, 2072 (Feb. 18, 2009)). Remarkably, these highly negatively charged structures (zeta potential <−30 mV) do not require cationic transfection materials or additional particle surface modifications and naturally enter all cell lines tested to date (over 50, including primary cells). Further work has shown the cellular uptake of these particles to be dependent upon DNA surface density; higher densities lead to higher levels of particle uptake (D. A. Giljohann et al., Nano Lett. 7, 3818 (December 2007)).

It has also previously been shown in WO 2008/098248 that polyvalent nucleic acid-gold nanoparticles that bind a target and are further labeled with a fluorophore associated with the binding polynucleotides can be used to determine the intracellular concentration of a target. In such nanoparticles, the signal from the fluorophore is quenched by the gold core. When they are contacted with a target molecule under conditions that allow association of the target molecule with the binding oligonucleotide on the nanoparticle, the fluorophore is released and a signal is generated that is proportional to the intracellular concentration of said target molecule.

The use of polyvalent nucleic acid-nanoparticles in vitro and in vivo has been questioned because of their intense coloration (in the case of gold), concerns of toxicity (especially for semiconductor nanoparticles), and unknown long-term biological/environmental interactions.

Hollow nanoconjugates have attracted significant interest in recent years due to their unique chemical, physical, and biological properties, which suggest a wide range of applications in drug/gene delivery (Shu et al., Biomaterials 31: 6039 (2010); Kim et al., Angew. Chem. Int. Ed. 49: 4405 (2010); Kasuya et al., In Meth. Enzymol.; Nejat, D., Ed.; Academic Press: 2009; Vol. Volume 464, p 147), imaging (Sharma et al., Contrast Media Mol. Imaging 5: 59 (2010); Tan et al., J. Chem. Commun. 6240 (2009)), and catalysis (Choi et al., Chem. Phys. 120: 18 (2010)).

A variety of methods have been developed to synthesize these structures based upon emulsion polymerizations (Anton et al., J. Controlled Release 128: 185 (2008); Landfester et al., J. Polym. Sci. Part A: Polym. Chem. 48: 493 (2010); Li et al., J. Am. Chem. Soc. 132: 7823 (2010)), layer-by-layer processes (Kondo et al., J. Am. Chem. Soc. 132: 8236 (2010)), crosslinking of micelles (Turner et al., Nano Lett. 4: 683 (2004); Sugihara et al., Angew. Chem. Int. Ed. 49: 3500 (2010); Moughton et al., Soft Matter 5: 2361 (2009)), molecular or nanoparticle self-assembly (Kim et al., Angew. Chem. Int. Ed. 46: 3471 (2007); Kim et al., J. Am. Chem. Soc. 132(28): 9908-19 (2010)), and sacrificial template techniques (Réthoré et al., Small 6: 488 (2010)).

Among them, the templating method is particularly powerful in that it transfers the ability to control the size and shape of the template to the product, for which desired homogeneity and morphology can be otherwise difficult to achieve. In a typical templated synthesis, a sacrificial core is chosen, upon which preferred materials containing latent crosslinking moieties are coated. Following the stabilization of the coating through chemical crosslinking, the template is removed, leaving the desired hollow nanoparticle. This additional crosslinking step can be easily achieved for compositionally simple molecules, such as poly(acrylic acid) or chitosan (Cheng et al., J. Am. Chem. Soc. 128: 6808 (2006); Hu et al., Biomacromolecules 8: 1069 (2007)). However, for systems containing sensitive and/or biologically functional structures, conventional crosslinking chemistries may not be sufficiently orthogonal to prevent the loss of their activity.

There remains a need for nanoconjugates which are more compatible with cells and can be used in vitro and in vivo diagnostics and therapeutics.

### BRIEF SUMMARY

One embodiment of the present invention is a structure formed from crosslinked polynucleotides, comprising a plurality of crosslinkable polynucleotides that are crosslinked; where a subset of the crosslinkable polynucleotides are binding polynucleotides that are sufficiently complementary to a target to allow them to hybridize under physiological conditions; a plurality of one member of a signal/quencher pair bound to the structure; and a plurality of signaling moieties hybridized to at least some of the binding polynucleotides in the structure, where each signaling moiety comprise the other member of the signal/quencher pair attached to a signal polynucleotide which is sufficiently complementary to the binding polynucleotide to allow it to hybridize under physiological conditions, where the signal group detectably changes when one or more of the signal polynucleotides is not hybridized to binding polynucleotide in the structure. In another embodiment, the crosslinkable polynucleotides are crosslinked through the spacer.

The structures of the present invention can be used to monitor binding of target polynucleotides in a cell in vitro or in vivo. The structures of the present invention can also be used in compositions, especially pharmaceutical compositions.

The structures of the present invention possess one of more unique properties compared to prior gold nanoparticles including enhanced cellular uptake, high bioactivity, and nuclease resistance.

### BRIEF DESCRIPTION OF THE DRAWINGS

FIG. 1 is an illustration of a structure of the present invention.

FIG. 2A is an IR spectra of PNANs synthesized from a sequence containing only T bases (10 modified with alkyne and 10 unmodified). 2B is an expanded view of the boxed area in 2A. Alkyne CEO stretching at 2115 cm−1 observed in the free DNA strand (black trace) disappears after the AuNP-catalyzed crosslinking process (grey trace).

FIG. 3 are ¹³C NMR spectra for free DNA and PNANs in D20. The DNA consisted of only T bases (vide infra). About 500 mL AuNP solution (5 nm, 3.95×1013 particles/mL, Ted Pella) was used to prepare PNANs for NMR studies. Resonances corresponding to the propargyl ether group (80.2, 76.7, 66.8, and 58.4 ppm) disappear after the catalytic crosslinking, and resonances corresponding to alcohol (58.8 ppm) and acetal crosslinks (70.3 ppm) arise.

FIG. 4 illustrates the dynamic light scattering measurement of number-averaged hydrodynamic diameters of AuNPs, AuNP-DNA conjugates and PNANs. Two series of particles, each based 10 nm or 30 nm citrate-capped AuNPs, are shown. Upon DNA adsorption, nanoparticle size increase consistently by about 14 nm in diameter for both 10 and 30 nm AuNP cores, which is expected from the length of the DNA strand (6.8 nm). When the AuNP core is removed, PNANs expand by ca. 7 nm.

FIG. 5 are TEM images of PNANs synthesized from (A, B) 10 and (C, D) 30 nm AuNP cores. Samples for TEM are prepared by drop-casting a PNAN solution on a plasma-treated carbon grid, followed by negative staining using uranyl acetate (2% wt).

FIG. 6 is an illustration of an experiment for determining the minimal length of the alkyne-modified region for sufficient crosslinking and successful PNAN formation. DNA strands, each having 1, 3, 5, 7, 9, 10 alkyne-modified T bases were assembled onto 10 nm AuNPs at equal density. Products were analyzed by 1% agarose gel electrophoresis. Products showing a minimal of 10 alkynes are preferable for complete PNAN formation.

FIG. 7 is a schematic of the synthesis of a structure using polyvalent propargyl ethers.

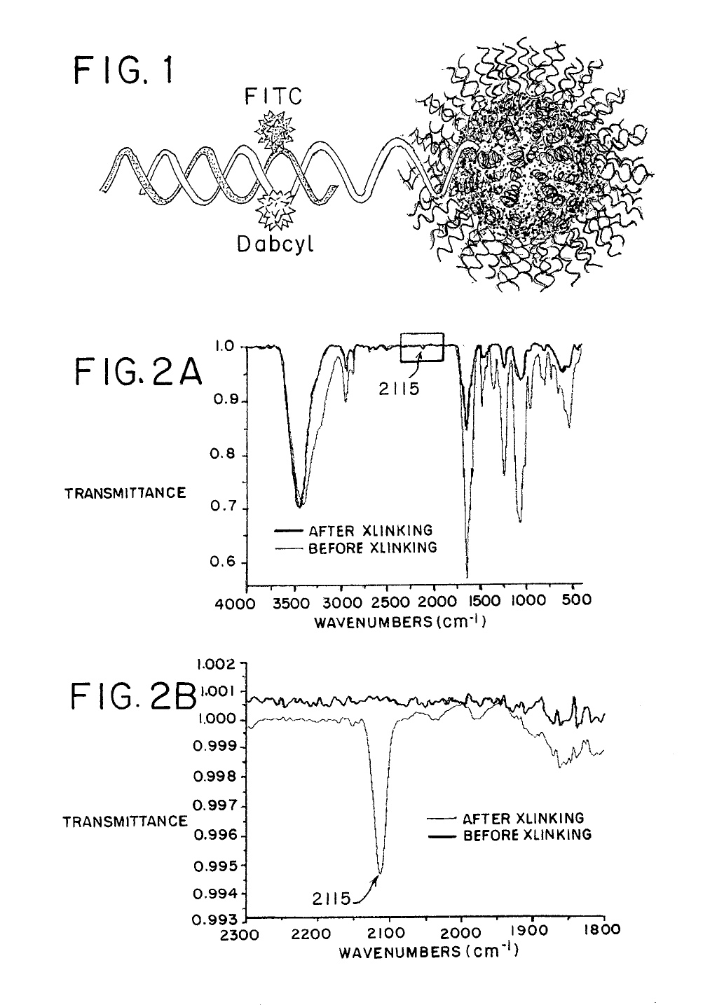

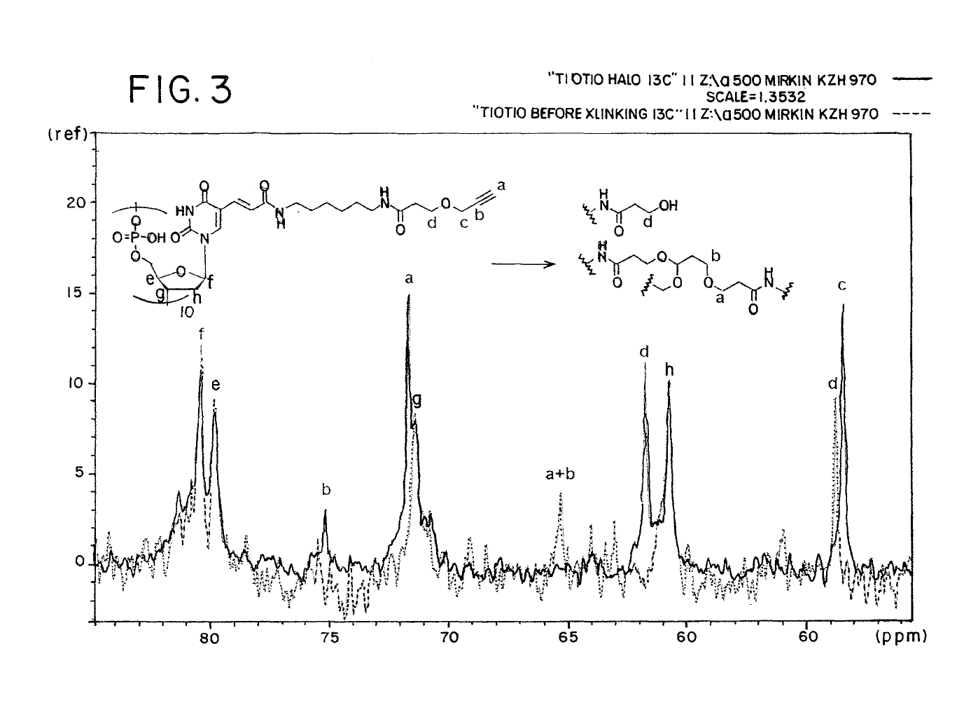

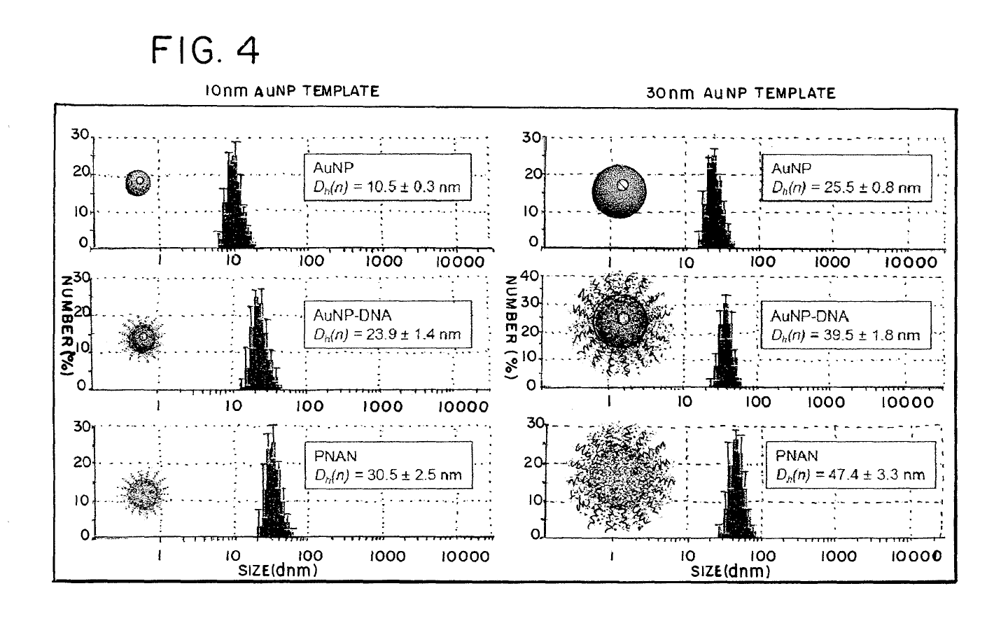

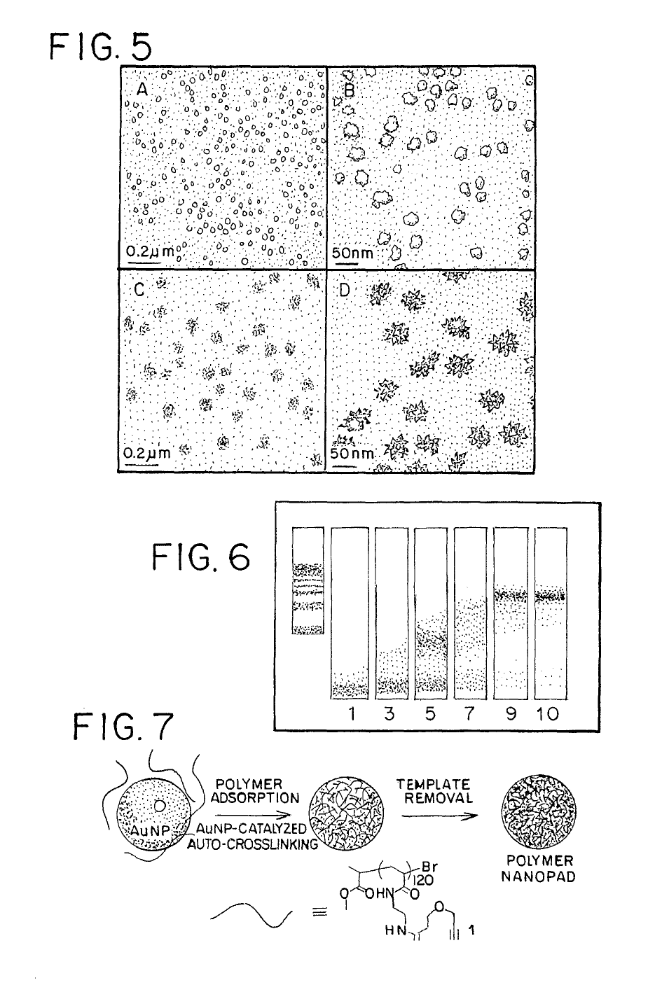

### DETAILED DESCRIPTION OF THE DRAWINGS AND THE PRESENTLY PREFERRED EMBODIMENTS

The Structure

The present invention provides a structure (or nanoconjugate) comprising a structure formed from crosslinked polynucleotides, where a subset of the crosslinkable polynucleotides are binding polynucleotides that are sufficiently complementary to a target to allow them to hybridize; a plurality of a first member of a signal/quencher pair bound to the structure; a plurality of signaling moieties hybridized to at least some of the binding polynucleotides in the structure, where each signaling moiety comprise the other member of the signal/quencher pair attached to a signal polynucleotide which is sufficiently complementary to the binding polynucleotide to allow it to hybridize, where the signal group detectably changes when one or more of the signal polynucleotides is not hybridized to binding polynucleotide in the structure.

In the structure, the crosslinked polynucleotides can be identical or different. In the structure, the binding polynucleotides can be identical or different. In the structure, the signal polynucleotides can be identical or different. Combinations, where all crosslinkable, binding and signaling polynucleotides are identical or where various subsets are different are contemplated, including (1) at least two different crosslinkable polynucleotides are combined with binding and signaling polynucleotides that are all identical, (2) all crosslinkable and binding polynucleotides are identical and at least two signaling polynucleotides are different, (3) all crosslinkable and signaling polynucleotides are identical and at least two binding polynucleotides are different, (4) at least two different crosslinkable polynucleotides are combined with two different binding and/or signaling polynucleotides, (5) at least two different signaling polynucleotides are combined with at least two different crosslinkable and/or binding polynucleotides, (6) at least two different binding polynucleotides are combined with at least tow different crosslinkable and/or signaling polynucleotides, etc.

The shape of the structure is determined by the surface used in its production, and optionally by the polynucleotides used in its production as well as well the degree and type of crosslinking between and among the polynucleotides. The surface is in various aspects planar or three dimensional. Necessarily a planar surface will give rise to a planar structure and a three dimensional surface will give rise to a three dimensional shape that mimics the three dimensional surface. When the surface is removed, a structure formed with a planar surface will still be planar, and a structure formed with a three dimensional surface will have the shape of the three dimensional surface and will be hollow.

Depending on the degree of crosslinking and the amount of starting polynucleotides, the structures provided are contemplated to have varying densities. Thus, the surface can be completely covered with crosslinked polynucleotides, or in an alternative aspects, significantly covered with crosslinked polynucleotides, or sparsely covered with the crosslinked polynucleotides. The density of coverage of the surface can be even over the entire surface or uneven over the surface.

The density of the crosslinked polynucleotides, along with the evenness or lack of evenness of the density over the surface will determine the porosity of the structure. In various aspects, the porosity determines the ability of the structure to entrap additional, non-structural agents, as discussed below, in the interior of the structure after the surface is removed.

Structures of the invention have a density of polynucleotides on the surface of the structure that is sufficient to result in cooperative behavior between structures and between polynucleotides in a single structure. In another aspect, the cooperative behavior between the structures increases the resistance of the polynucleotides to degradation. In one aspect, the uptake of structures by a cell is influenced by the density of polynucleotides. In general, a higher density of polynucleotides on the structure will increase uptake of the structure by a cell.

A surface density adequate to make the structures stable can be determined empirically. Broadly, the smaller the polynucleotide, the higher the surface density of that polynucleotide that can be. Generally, a surface density of at least 2 pmol/cm² will be adequate to provide stable structures. Alternatively, the surface density is at least 2 pmol/cm2, at least 3 pmol/cm2, at least 4 pmol/cm2, at least 5 pmol/cm2, at least 6 pmol/cm2, at least 7 pmol/cm2, at least 8 pmol/cm2, at least 9 pmol/cm2, at least 10 pmol/cm2, at least about 15 pmol/cm2, at least about 20 pmol/cm2, at least about 25 pmol/cm2, at least about 30 pmol/cm2, at least about 35 pmol/cm2, at least about 40 pmol/cm2, at least about 45 pmol/cm2, at least about 50 pmol/cm2, at least about 75 pmol/cm2, at least about 100 pmol/cm2, at least about 250 pmol/cm2, at least about 500 pmol/cm2, at least about 750 pmol/cm2, at least about 1000 pmol/cm2 or more.

In various aspects, the structures of the present invention range in size from about 1 nm to about 250 nm in mean diameter. Alternatively, they range in size from, about 1 nm to about 1 nm to about 200 nm, about 1 nm to about 100 nm, about 1 nm to about 50 nm, about 1 nm to about 25 nm, about 1 nm to about 10 nm. In other aspects, the size of the nanosurface is from about 5 nm to about 150 nm (mean diameter), from about 5 to about 50 nm, from about 10 to about 30 nm.

Nanosurfaces of larger diameter are, in some aspects, contemplated to be templated with a greater number of polynucleotides (Hurst et al., Analytical Chemistry 78(24): 8313-8318 (2006)) during structure production. The number of polynucleotides used in the production of a structure is from about 10 to about 25,000 polynucleotides per structure. Alternatively, the number is from about 50 to about 10,000 polynucleotides per structure, from about 200 to about 5,000 polynucleotides per structure.

It is also contemplated that polynucleotide surface density modulates the stability of the polynucleotide associated with the structure. Thus, in one embodiment, a structure comprising a polynucleotide is provided wherein the polynucleotide has a half-life that is at least substantially the same as the half-life of an identical polynucleotide that is not part of a structure. In other embodiments, the polynucleotide associated with the nanosurface has a half-life that is about 5% greater to about 1,000,000-fold greater or more than the half-life of an identical polynucleotide that is not part of a structure.

Polynucleotides include short internal complementary polynucleotides (sicPN), DNA, RNA (including siRNA), LNA, modified forms and combinations thereof. The polynucleotide can be double stranded or single stranded. As used herein, polynucleotide is interchangeable with oligonucleotide.

Crosslinkable, binding and signaling polynucleotides can each independently be of a length ranging from about 5 nucleotides to about 100 nucleotides, from about 5 to about 90 nucleotides, from about 5 to about 80 nucleotides, about 5 to about 70 nucleotides, about 5 to about 60 nucleotides, about 5 to about 50 nucleotides about 5 to about 45 nucleotides, about 5 to about 40 nucleotides, about 5 to about 35 nucleotides, about 5 to about 30 nucleotides, about 5 to about 25 nucleotides, about 5 to about 20 nucleotides, about 5 to about 15 nucleotides, about 5 to about 10 nucleotides, and all polynucleotides intermediate of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides are contemplated.

The polynucleotides in the structure can contain a single sequence, multiple copies of a single sequence, or multiple copies of different sequences. When multiple copies are present, two, three, four, five, six, seven eight, nine, ten or more can be present. When multiple copies of different sequences are present, they can be in repeating patterns.

Polynucleotides can be composed of naturally-occurring nucleotide and/or non-naturally-occurring nucleotides (including modified nucleotides). Naturally occurring nucleotides include adenine (A), guanine (G), cytosine (C), thymine (T) and uracil (U). Non-naturally occurring nucleotides include, for example, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr- iazolopyridine, isocytosine, isoguanine, inosine, etc. Nucleotides include not only the known purine and pyrimidine heterocycles, but also heterocyclic analogues and tautomers thereof.

Further naturally and non-naturally occurring nucleobases include those described in U.S. Pat. No. 5,432,272 (Benner et al.), S M Freier et al., Nucleic Acids Research, 1997, 25: 4429-4443, U.S. Pat. No. 3,687,808 (Merigan, et al.), Sanghvi in Chapter 15 of Antisense Research and Application, Ed. S. T. Crooke and B. Lebleu, CRC Press, 1993, Englisch et al., Angewandte Chemie, International Edition, 1991, 30: 613-722, the Concise Encyclopedia of Polymer Science and Engineering, J. I. Kroschwitz Ed., John Wiley & Sons, 1990, pages 858-859, Cook, Anti-Cancer Drug Design, 1991, 6:585-607, each of which are hereby incorporated by reference in their entirety. In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include pyrrole, diazole or triazole derivatives, etc.

Modified nucleotides are described in EP 1 072 679 and WO 97/12896, the disclosures of which are incorporated herein by reference. Modified nucleotides include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox- azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993.

Certain bases are known to increase binding affinity, including 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

One example of a modified form of a polynucleotide is a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other modified forms include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of modified forms of polynucleotides include those containing modified backbones or non-natural internucleoside linkages. Polynucleotides having modified backbones include those that retain a phosphorus atom in the backbone and those that do not have a phosphorus atom in the backbone. Modified polynucleotides that do not have a phosphorus atom in their internucleoside backbone are considered to be within the meaning of “polynucleotide.”

Modified polynucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH-O-CH₂—, —CH₂-N(CH₃)-O-CH₂—, —CH₂-O-N(CH₃)-CH₂—, —CH₂-N(CH₃)-N(CH₃)-CH₂- and —O-N(CH₃)-CH₂-CH₂- described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the crosslink between two crosslinkable polynucleotides consists of 2 to 4, desirably 3, groups/atoms selected from —CH₂—, —O—, —S—, —NRH—, >C=O, >C=NRH, >C=S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)-, where RH is selected from hydrogen and C₁₋₄-alkyl, and R″ is selected from C₁₋₆-alkyl and phenyl. Illustrative examples of such linkages are —CH₂-CH₂-CH₂—, —CH₂-CO-CH₂—, —CH₂-CHOH-CH₂—, —O-CH₂-O—, —O-CH₂-CH₂-, —O-CH₂-CH=, —CH₂-CH₂-O—, —NRH-CH₂-CH₂—, —CH₂-CH₂-NRH—, —CH₂-NRH-CH₂—, —O-CH₂-CH₂-NRH—, —NRH-CO-O—, —NRH-CO-NRH—, —NRH-CS-NRH—, —NRH-C(=NRH)-NRH—, —NRH-CO-CH₂-NRH-O-CO-O—, —O-CO-CH₂-O—, —O-CH₂-CO-O—, —CH₂-CO-NRH—, —O-CO-NRH—, —NRH-CO-CH₂—, —O-CH₂-CO-NRH—, —O-CH₂-CH₂-NRH—, —CH=N-O—, —CH₂-NRH-O—, —CH₂-O-N=, —CH₂-O-NRH—, —CO-NRH-CH₂—, —CH₂-NRH-O—, —CH₂-NRH-CO—, —O-NRH-CH₂—, —O-NRH, —O-CH₂-S—, —S-CH₂-O—, —CH₂-CH₂-S—, —O-CH₂-CH₂-S—, —S-CH2-CH=, —S-CH₂-CH₂—, —S-CH₂-CH₂-O—, —S-CH₂-CH₂-S—, —CH₂-S-CH₂—, —CH₂-SO-CH₂—, —CH₂-SO₂-CH₂—, —O-SO-O—, —O-S(O)₂-O—, —O-S(O)2-CH₂—, —O-S(O)₂-NRH—, —NRH-S(O)₂-CH₂—, —O-S(O)₂-CH₂—, —O-P(O)₂-O—, —O-P(O,S)-O—, —O-P(S)₂-O—, —S-P(O)₂-O—, —S-P(O,S)-O—, —S-P(S)₂-O—, —O-P(O)₂-S—, —O-P(O,S)-S—, —O-P(S)₂-S—, —S-P(O)₂-S—, —S-P(O,S)-S—, —S-P(S)₂-S—, —O-PO(R″)-O—, —O-PO(OCH₃)-O—, —O-PO(OCH₂CH₃)-O—, —O-PO(OCH₂CH₂S-R)-O—, —O-PO(BH₃)-O—, —O-PO(NHRN)-O—, —O-P(O)₂-NRH H—, —NRH-P(O)₂-O—, —O-P(O,NRH)-O—, —CH₂-P(O)₂-O—, —O-P(O)₂-CH₂-, and —O-Si(R″)₂-O-; among which —CH₂-CO-NRH—, —CH₂-NRH-O—, —S-CH₂-O—, —O-P(O)₂-O-O-P(-O,S)-O—, —O-P(S)₂-O—, —NRH P(O)₂-O—, —O-P(O,NRH)-O—, —O-PO(R″)-O—, —O-PO(CH₃)-O-, and —O-PO(NHRN)-O-, where RH is selected form hydrogen and C1-4-alkyl, and R″ is selected from C1-6-alkyl and phenyl, are contemplated. Further illustrative examples are given in Mesmaeker et al., 1995, Current Opinion in Structural Biology, 5: 343-355 and Susan M. Freier and Karl-Heinz Altmann, 1997, Nucleic Acids Research, 25: 4429-4443.

Still other modified forms of polynucleotides are described in detail in U.S. Publication No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other embodiments include O[(CH2)nO]mCH3, O(CH2)nOCH3, O(CH2)nNH2, O(CH2)nCH3, O(CH2)nONH2, and O(CH2)nON[(CH2)nCH3]2, where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O-CH2CH2OCH3, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH2)20N(CH3)2 group, also known as 2′-DMAOE, and 2′-dimethylaminoethoxyethoxy (also known in the art as 2′-O-dimethyl-amino-ethoxy-ethyl or 2′-DMAEOE), i.e., 2′-O-CH2-O-CH2-N(CH3)2.

Still other modifications include 2′-methoxy (2′-O-CH3), 2′-aminopropoxy (2′-OCH2CH2CH2NH2), 2′-allyl (2′-CH2-CH=CH2), 2′-O-allyl (2′-O-CH2-CH=CH2) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (-CH2-)n group bridging the 2′ oxygen atom and the 4′ carbon atom wherein n is 1 or 2. LNAs and preparation thereof are described in WO 98/39352 and WO 99/14226, the disclosures of which are incorporated herein by reference.

In some embodiments, the disclosure contemplates that a polynucleotide used in the production of a structure is RNA. When RNA is part of a structure, the RNA is, in some aspects, comprised of a sequence that is sufficiently complementary to a target sequence of a polynucleotide such that hybridization of the RNA polynucleotide attached to a structure and the target polynucleotide takes place. In aspects wherein a sicPN is utilized, hybridization of the RNA polynucleotide that is part of the structure and the target polynucleotide associates the target polynucleotide with the structure, causing displacement and/or release of a sicPN as described herein. The RNA in various aspects is single stranded or double stranded, as long as the double stranded molecule also includes, in some aspects, a single strand sequence that hybridizes to a single strand sequence of the target polynucleotide.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Patent No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

The shape of each structure in the plurality is determined in part by the surface used in its production, and in part by the polynucleotides used in its production.

The surface is in various aspects planar or three dimensional. Thus, in various aspects, the surface is a nanoparticle.

In general, nanosurfaces include any compound or substance with a high loading capacity for a polynucleotide to effect the production of a structure as described herein, including for example and without limitation, a metal, a semiconductor, an insulator particle, and a dendrimer.

Thus, nanosurfaces are contemplated which comprise a variety of inorganic materials including, but not limited to, metals, semi-conductor materials or ceramics as described in U.S. Publication No. 2003/0147966. For example, metal-based nanosurfaces include those described herein. Ceramic nanosurface materials include, but are not limited to, brushite, tricalcium phosphate, alumina, silica, and zirconia. Organic materials from which nanosurfaces are produced include carbon. Polymeric nanosurfaces include those formed from polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also contemplated for use as nanosurfaces.

In one embodiment, the nanosurface is metallic, and in various aspects, the nanosurface is a colloidal metal. Thus, in various embodiments, nanosurfaces useful in the practice of the methods include metal (including gold, silver, platinum, aluminum, palladium, copper, cobalt, iron, indium, nickel, or any other metal amenable to nanosurface formation), semiconductor (including for example and without limitation, CdSe, CdS, and CdS or CdSe coated with ZnS) and magnetic (for example, ferromagnetite) colloidal materials. Other nanosurfaces include, also without limitation, ZnS, ZnO, Ti, TiO2, Sn, SnO2, Si, SiO2, Fe, Fe+4, Fe3O4, Fe2O3, Ag, Cu, Ni, Al, steel, cobalt-chrome alloys, Cd, titanium alloys, AgI, AgBr, HgI2, PbS, PbSe, ZnTe, CdTe, In2S3, In2Se3, Cd3P2, Cd3As2, InAs, and GaAs. Methods of making ZnS, ZnO, TiO2, AgI, AgBr, HgI2, PbS, PbSe, ZnTe, CdTe, In2S3, In2Se3, Cd3P2, Cd3As2, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); Ushida et al., J. Phys. Chem., 95, 5382 (1992).

In practice, structures and methods are provided using any suitable nanosurface that does not interfere with crosslinking. The size, shape and chemical composition of the nanosurface contribute to the properties of the resulting structure. These properties include for example, optical properties, optoelectronic properties, electrochemical properties, electronic properties, stability in various solutions, magnetic properties, and pore and channel size variation. The use of mixtures of surfaces having different sizes, shapes and/or chemical compositions, as well as the use of nanosurfaces having uniform sizes, shapes and chemical composition, is contemplated.

In one embodiment the nanosurface is a nanoparticles including, for example and without limitation, nanoparticles, aggregate particles, isotropic (such as spherical particles) and anisotropic particles (such as non-spherical rods, tetrahedral, prisms) and core-shell particles such as the ones described in U.S. Pat. No. 7,238,472 and WO 2002/096262, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine) are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers)

Also as described in U.S. publication No 20030147966, nanoparticles comprising materials described herein are available commercially from, for example, Ted Pella, Inc. (gold), Amersham Corporation (gold) and Nanoprobes, Inc. (gold), or they can be produced from progressive nucleation in solution (e.g., by colloid reaction), or by various physical and chemical vapor deposition processes, such as sputter deposition. See, e.g., Hayashi, (1987) Vac. Sci. Technol. July/August 1987, A5(4):1375-84; Hayashi, (1987) Physics Today, December 1987, pp. 44-60; MRS Bulletin, January 1990, pgs. 16-47.

As further described in U.S. Publication No. 2003/0147966, nanoparticles contemplated are produced using HAuCl₄ and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various structures and size ranges are available, for example, from Vector Laboratories, Inc. of Burlingame, Calif.

As described herein, in various aspects the structures provided by the disclosure are hollow. The porosity and/or rigidity of a hollow structure depends in part on the density of the crosslinked polynucleotides that form the structure. In general, a lower density of crosslinked polynucleotides results in a more porous structure, while a higher density of crosslinked polynucleotides results in a more rigid structure. Porosity and density of a hollow structure also depends on the degree and type of crosslinking between polynucleotides.

In some aspects, a hollow structure is produced which is then loaded with a desirable additional agent, and the structure is then covered with a coating to prevent the escape of the additional agent. The coating, in some aspects, is also an additional agent and is described in more detail below.

Crosslinkable Polynucleotides

Each crosslinkable polynucleotide, prior to crosslinking, comprises at least one moiety that can crosslink. When multiple crosslinking moieties are present on a single polynucleotide, some intramolecular crosslinking may occur but is not detrimental to the final structure. The moiety can either be present in the polynucleotide (for example, in a modified base) or can be present in a spacer that is covalently attached to the polynucleotide or present in some crosslinkable polynucleotides and some spacers.

The crosslinking moieties in different crosslinked polynucleotides or in polynucleotides with multiple crosslinking moieties can be the same or different. The crosslinking moiety can be similarly located in each polynucleotide, which under certain conditions orients all of the polynucleotides in the same direction. In another aspect, the crosslinking moiety is located in different positions in the polynucleotide, which under certain conditions can provide mixed orientation of the polynucleotides after crosslinking.

In some aspects, the present structures allow for efficient uptake of the structure into cells. In various aspects, the crosslinkable polynucleotide comprises a nucleotide sequence that allows increased uptake efficiency of the structure. As used herein, “efficiency” refers to the number or rate of uptake of structures in/by a cell. Because the process of structures entering and exiting a cell is a dynamic one, efficiency can be increased by taking up more structures or by retaining those structures that enter the cell for a longer period of time. Similarly, efficiency can be decreased by taking up fewer structures or by retaining those structures that enter the cell for a shorter period of time.

The nucleotide sequence can be any nucleotide sequence that is desired may be selected for, in various aspects, increasing or decreasing cellular uptake of a structure or gene regulation. The nucleotide sequence, in some aspects, comprises a homopolymeric sequence which affects the efficiency with which the nanoparticle to which the polynucleotide is attached is taken up by a cell. Accordingly, the homopolymeric sequence increases or decreases the efficiency. It is also contemplated that, in various aspects, the nucleotide sequence is a combination of nucleobases, such that it is not strictly a homopolymeric sequence. For example and without limitation, in various aspects, the nucleotide sequence comprises alternating thymidine and uridine residues, two thymidines followed by two uridines or any combination that affects increased uptake is contemplated by the disclosure. In some aspects, the nucleotide sequence affecting uptake efficiency is included as a domain in a polynucleotide comprising additional sequence. This “domain” would serve to function as the feature affecting uptake efficiency, while the additional nucleotide sequence would serve to function, for example and without limitation, to regulate gene expression. In various aspects, the domain in the polynucleotide can be in either a proximal, distal, or center location relative to the structure. It is also contemplated that a polynucleotide comprises more than one domain.

The homopolymeric sequence, in some embodiments, increases the efficiency of uptake of the structure by a cell. In some aspects, the homopolymeric sequence comprises a sequence of thymidine residues (polyT) or uridine residues (polyU). In further aspects, the polyT or polyU sequence comprises two thymidines or uridines. In various aspects, the polyT or polyU sequence comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 125, about 150, about 175, about 200, about 250, about 300, about 350, about 400, about 450, about 500 or more thymidine or uridine residues.

In some embodiments, the structure comprises a crosslinkable polynucleotide that comprises a homopolymeric sequence is taken up by a cell with greater efficiency than a structure comprising the same polynucleotide but lacking the homopolymeric sequence. In various aspects, a structure comprising a polynucleotide that comprises a homopolymeric sequence is taken up by a cell about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold or higher, more efficiently than a structure comprising the same polynucleotide but lacking the homopolymeric sequence.

In other aspects, the domain is a phosphate polymer (C3 residue). In some aspects, the domain comprises a phosphate polymer (C3 residue) that is comprised of two phosphates. In various aspects, the C3 residue comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 125, about 150, about 175, about 200, about 250, about 300, about 350, about 400, about 450, about 500 or more phosphates.

In some embodiments, it is contemplated that a structure comprising a polynucleotide which comprises a domain is taken up by a cell with lower efficiency than a structure comprising the same polynucleotide but lacking the domain. In various aspects, a structure comprising a polynucleotide which comprises a domain is taken up by a cell about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold or higher, less efficiently than a structure comprising the same polynucleotide but lacking the domain.

Crosslinking moieties contemplated by the disclosure include but are not limited to an amine, amide, alcohol, ester, aldehyde, ketone, thiol, disulfide, carboxylic acid, phenol, imidazole, hydrazine, hydrazone, azide and an alkyne.

In one embodiment, an alkyne is associated with a crosslinkable polynucleotide through a degradable moiety. For example, the alkyne is attached to an acid-labile moiety that degrades upon entry into a cell.

In some aspects, the nanosurface acts as a catalyst for the crosslinking moieties. Under appropriate conditions, contacting a crosslinking moiety with the surface will activate crosslinking, thereby initiating crosslinking between crosslinkable polynucleotides. In one specific aspect, the crosslinking moiety is an alkyne and the surface is comprised of gold. In this aspect, and as described herein, the gold surface acts as a catalyst to activate an alkyne crosslinking moiety.

Production methods are also contemplated wherein a chemical is used to crosslink the crosslinkable polynucleotides. Polynucleotides contemplated for use in the methods include those associated with a structure through any means. Regardless of the means by which the polynucleotide is associated with the structure, association in various aspects is effected through a 5′ linkage, a 3′ linkage, some type of internal linkage, or any combination of these attachments and depends on the location of the crosslinking moiety in the polynucleotide. By way of example, a crosslinking moiety on the 3′ end of a polynucleotide means that the polynucleotide will associate with the structure at its 3′ end.

In various aspects, the crosslinking moiety is located in a spacer. A spacer is described herein above, and it is contemplated that a nucleotide in the spacer comprises a crosslinking moiety. In further aspects, a nucleotide in the spacer comprises more than one crosslinking moiety, and the more than one crosslinking moieties are either the same or different. In addition, each nucleotide in a spacer can comprise one or more crosslinking moieties, which can either be the same or different.

In some embodiments, the polynucleotide does not comprise a spacer. In these aspects, the polynucleotide comprises one or more crosslinking moieties along its length. The crosslinking moieties can be the same or different, and each nucleotide in the polynucleotide can comprise one or more crosslinking moieties, and these too can either be the same or different.

The crosslinkable polynucleotides comprise from about 1 to about 500 crosslinking moieties or from about 1 to about 100 or from about 5 to about 50 or from about 10 to about 30 or from about 10 to about 20 crosslinking moieties. In various embodiments, the polynucleotide comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more crosslinking moieties. In aspects wherein the spacer comprises more than one crosslinking moiety, the moieties can all be the same or they can be different, and any combination of crosslinking moieties may be used.

In one aspect, the crosslinking moiety is located in the same position in each polynucleotide, which under certain conditions orients all of the polynucleotides in the same direction. In some aspects, the direction is such that the 5′ and 3′ ends of a polynucleotide are diametrically opposed to each other. In these aspects, the spacer end will be more “proximal” with respect to the structure surface, while the opposite end will be more “distal” with respect to the structure surface. With respect to “proximal” and “distal” and their relationship to the structure surface, it will be understood that the location is determined when the surface is present, and prior to its optional at least partial removal. The orienting of polynucleotides in the same direction in a structure is useful, for example and without limitation, when a binding polynucleotide is to be hybridized to a target since the structure provides a polyvalent network of polynucleotides that are positioned to recognize and associate with the target.

In another aspect, the crosslinking moiety is located in different positions in the polynucleotides, which under certain conditions can provide mixed orientation of the polynucleotides after crosslinking.

Spacer

Optionally, the structure can further comprise a spacer. In one embodiment, the spacer can comprise one or more crosslinking moieties that facilitate the crosslinking of one crosslinkable polynucleotide to another polynucleotide (crosslinking between crosslinkable polynucleotides, binding polynucleotides or combinations thereof). In another embodiment, the spacer is used to increase the distance between the core of the structure and the crosslinkable and binding polynucleotides. When a structure is used for gene regulation, the spacer is generally designed to not directly participate in the gene regulation.

The spacer can be an organic moiety, a polymer (preferably water-soluble polymers), a polynucleotide, a polypeptide, an oligosaccharide, a carbohydrate, a lipid, or combinations thereof.

When the spacer is a polynucleotide, the length of the spacer is at least about 5 nucleotides, at least about 10 nucleotides, 10-30 nucleotides, or greater than 30 nucleotides. The spacer may have any sequence which does not interfere with the ability of the polynucleotides to become bound to the nanoparticles or to the target polynucleotide. The spacers should not have sequences complementary to each other or to that of the polynucleotides, but may be all or in part complementary to the target polynucleotide. In certain aspects, the bases of the polynucleotide spacer are all adenines, all thymines, all cytidines, all guanines, all uracils, or all some other modified base.

Au(I) and Au(III) ions and their complexes display remarkable alkynophilicity, and have been increasingly recognized as potent catalysts for organic transformations (Hashmi, Chem. Rev. 107: 3180-3211 (2007); Li et al., Chem. Rev. 108: 3239 (2008); Fürstner et al., Angew. Chem. Int. Ed. 46: 3410 (2007); Hashmi et al., Angew. Chem. Int. Ed. 45: 7896 (2006)). Recently, it has been demonstrated that Au(O) surfaces also adsorb terminal acetylene groups and form relatively densely packed and stable monolayers [Zhang et al., J. Am. Chem. Soc. 129: 4876 (2006)]. However, the type of interaction that exists between the alkyne and the gold surface is not well understood.

Moreover, it is not clear whether such interaction makes the acetylene group more susceptible to chemical reactions, such as nucleophilic additions typically observed with ionic gold-alkyne complexes. Bearing multiple side-arm propargyl ether groups, polymer 1 (FIG. 7) readily adsorbs onto citrate-stabilized 13 nm AuNPs prepared in an aqueous solution following the Turkevich-Frens method (Frens, Coll. Polym. Sci. 250: 736 (1972)). Excess polymer is removed by iterative centrifugation and subsequent resuspension steps. The resulting polymer-coated AuNPs exhibit a plasmon resonance at 524 nm characteristic of dispersed particles, and there is no evidence of aggregation even after 8 weeks of storage at room temperature. Therefore, even though 1 is a potential inter-particle crosslinking agent, it does not lead to aggregation of the AuNPs, a conclusion that was corroborated by Dynamic Light Scattering (DLS) and electron microscopy.

In one embodiment, the disclosure provides a method for synthesizing structures from a linear biomolecule bearing pendant propargyl ether groups (1), utilizing gold nanoparticles (AuNPs) as both the template for the formation of the shell and the catalyst for the crosslinking reaction (FIG. 7). No additional crosslinking reagents or synthetic operations are required. The reaction yields well-defined, homogeneous hollow structures when the nanosurface is removed after the polynucleotides are crosslinked.

In one embodiment, the crosslinkable polynucleotides each independently comprise an alkyne group. In another embodiment, the crosslinkable polynucleotides each are linked to a spacer comprising an alkyne group. In various embodiments, from 1 to 100 alkyne moieties are independently present on each crosslinkable polynucleotide. In further aspects, from about 5 to about 50 alkyne moieties or about 10 to about 20 alkyne moieties are present on a polynucleotide. In one aspect, 10 alkyne moieties are present on the polynucleotide. In further aspects, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more alkyne moieties are present on a polynucleotide.

In another embodiment, the alkyne moieties on the crosslinkable polynucleotide are on the 5′ end. In a further embodiment, the alkyne moieties on the crosslinkable polynucleotide are on the 3′ end. It is contemplated that in some aspects the alkyne moieties represent only a portion of the length of a crosslinkable polynucleotide. By way of example, if a crosslinkable polynucleotide is 20 nucleotides in length, then it is contemplated that the first 10 nucleotides (counting, in various aspects from either the 5′ or 3′ end) comprise an alkyne moiety. Thus, 10 nucleotides comprising an alkyne moiety out of a total of 20 nucleotides results in 50% of the nucleotides in a crosslinkable polynucleotide have an alkyne moiety. In various aspects it is contemplated that from about 0.01% to about 100% of the nucleotides in the crosslinkable polynucleotide are associated with an alkyne moiety. In further aspects, about 1% to about 70%, or about 2% to about 60%, or about 5% to about 50%, or about 10% to about 50%, or about 10% to about 40%, or about 20% to about 50%, or about 20% to about 40% of nucleotides in the crosslinkable polynucleotides are associated with an alkyne moiety.

Returning to methods of carrying out the crosslinking using a poly alkyne crosslinking approach, the following steps are involved. First, a solution of containing a nanosurface is prepared. The solution is brought into contact with a solution comprising polynucleotides comprising a poly-reactive group (Contacting step). Depending on the polyreactive group, an optional activation step is included (Activation step). The resulting mixture is then incubated to allow the crosslinking to occur (Incubation step), and is then isolated (Isolation step). The nanosurface is then dissolved (Dissolution step). When the nanosurface in on a nanoparticle, a hollow structure is created. Binding polynucleotides are then hybridized to signaling moieties (Labeling step).

The structures of the present invention can be prepared as described in the examples below. Briefly, the method involves contacting crosslinkable polynucleotides, including a subset of which are binding polynucleotides, with a gold structure, crosslinking the crosslinkable polynucleotides, dissolving the gold core and hybridizing signaling moieties to some or all of the binding polynucleotides.

Crosslinkable polynucleotides containing a poly-reactive group (either at a terminus, within a modified base) or crosslinkable polynucleotides and spacers with a poly-reactive group are contacted with a nanosurface in solution. The polyreactive group can be an alkyne, or the polyreactive group can be a light-reactive group, or a group that is activated upon, for example and without limitation, sonication or microwaves.

Regardless of the crosslinking strategy that is used, the amount of crosslinkable polynucleotides used depends on the desired properties of the resulting structure. A lower concentration of crosslinkable polynucleotides will result in a lower density on the nanosurface, which will result in a more porous structure. Conversely, a higher concentration of crosslinkable polynucleotides will result in a higher density on the nanosurface, which will result in a more rigid structure. A “lower density” is from about 2 pmol/cm² to about 100 pmol/cm². A “higher density” is from about 101 pmol/cm² to about 1000 pmol/cm².

When the polyreactive group present on the crosslinkable polynucleotides requires activation, the source of activation can be, without limitation, a laser (when the polyreactive group is light reactive), or sound (when the polyreactive group is activated by sonication), or a microwave (when the polyreactive group is activated by microwaves). In some embodiments, the nanosurface itself can activate the polyreactive groups present on the surface. In these embodiments, an activation step is not required.

Once the solution comprising the crosslinkable polynucleotides is brought into contact with the solution containing the nanosurface(s), the mixture is incubated to allow crosslinking to occur. Incubation can occur at a temperature from about 4° C. to about 50° C. The incubation is allowed to take place for a time for at least 1 minute, preferably from about 1 minute to about 48 hours or more.

Once the crosslinkable polynucleotides are crosslinked, the structure(s) can then be isolated. For isolation, the mixture is centrifuged, the supernatant is removed and the crosslinked structures are resuspended in an appropriate buffer. In various aspects, more than one centrifugation step may be carried out to further purify the crosslinked structures.

Dissolution of a nanosurface is within the ordinary skill in the art. In one embodiment, it is dissolved by using KCN in the presence of oxygen. Alternatively, iodine or Aqua regia is used to dissolve the nanosurface. In a preferred embodiment, the nanosurface comprises gold. When KCN is added to citrate stabilized gold surfaces, the color of the solution changes from red to purple, resulting from the destabilization and aggregation of the gold nanosurfaces. However, for polymer-coated gold nanosurfaces, the color slowly changes to a slightly reddish orange color during the dissolution process until the solution is clear.

The dissolution process can be visualized by transmission electron microscopy (TEM). As the outer layer of the gold nanosurface is partially dissolved, the protective shell mentioned above can be observed with uranyl-acetate staining of the TEM grid. Complete removal of the template affords structures that retain the size and shape of their gold nanosurface (template) in high fidelity.

Direct strand crosslinking (DSC) is a method whereby one or more nucleotides of a crosslinkable polynucleotide is modified with one or more crosslinking moieties that can be cross-linked through chemical means. The DSC method involves the modification of one or more nucleotides of the crosslinkable polynucleotides with a moiety that can be crosslinked through a variety of chemical means. In another embodiment, the one or more nucleotides that are modified are in the spacer.

Briefly, crosslinkable polynucleotides are synthesized that incorporate an amine-modified thymidine phosphoramidite (TN). The cross-linking efficiency will depend on the amount of modified bases in the polynucleotides, the spacers or both.

The strands may be crosslinked with the use of a homobifunctional cross-linker like Sulfo-EGS, which has two amine reactive NHS-ester moieties. Although amines are contemplated for use in one embodiment, this design is compatible with many other reactive groups (for example and without limitation, amines, amides, alcohols, esters, aldehydes, ketones, thiols, disulfides, carboxylic acids, phenols, imidazoles, hydrazines, hydrazones, azides, and alkynes).

An additional method, called surface assisted crosslinking (SAC), comprises a mixed monolayer of crosslinkable polynucleotides and reactive thiolated molecules that are assembled on the nanosurface and crosslinked together.

The chemical that causes crosslinking of the crosslinkable polynucleotides include without limitation disuccinimidyl glutarate, disuccinimidyl suberate, bis[sulfosuccinimidyl] suberate, tris-succinimidyl aminotriacetate, succinimidyl 4-hydrazinonicotinate acetone hydrazone, succinimidyl 4-hydrazidoterephthalate hydrochloride, succinimidyl 4-formylbenzoate, dithiobis[succinimidyl propionate], 3,3″-dithiobis[sulfosuccinimidylpropionate], disuccinimidyl tartarate, bis[2-(succinimidooxycarbonyloxy)ethyl]sulfone, ethylene glycol bis[succinimidylsuccinate], ethylene glycol bis[sulfosuccinimidylsuccinate], dimethyl adipimidate•2 hcl, dimethyl pimelimidate•2 HCl, dimethyl suberimidate•2 HCl, 1,5-difluoro-2,4-dinitrobenzene, β-[tris(hydroxymethyl) phosphino] propionic acid, bis-maleimidoethane, 1,4-bismaleimidobutane, bismaleimidohexane, tris[2-maleimidoethyl]amine, 1,8-bis-maleimido-diethyleneglycol, 1,11-bis-maleimido-triethyleneglycol, 1,4 bismaleimidyl-2,3-dihydroxybutane, dithio-bismaleimidoethane, 1,4-di-[3″-(2″-pyridyldithio)-propionamido]butane, 1,6-hexane-bis-vinylsulfone, bis-[b-(4-azidosalicylamido)ethyl]disulfide, N-(α-maleimidoacetoxy) succinimide ester, N-[R-maleimidopropyloxy]succinimide ester, N-[γ-maleimidobutyryloxy]succinimide ester, N-[γ-maleimidobutyryloxy]sulfosuccinimide ester, m-maleimidobenzoyl-N-hydroxysuccinimide ester, m-maleimidobenzoyl-N-hydroxysulfosuccinimide ester, succinimidyl 4-[N-maleimidomethyl]cyclohexane-1-carboxylate, sulfosuccinimidyl 4-[N-maleimidomethyl]cyclohexane-1-carboxylate, N-ε-maleimidocaproyloxy]succinimide ester, N-ε-maleimidocaproyloxy]sulfosuccinimide ester, succinimidyl 4-[p-maleimidophenyl]butyrate, sulfosuccinimidyl 4-[p-maleimidophenyl]butyrate, succinimidyl-6-[β-maleimidopropionamido]hexanoate, succinimidyl-4-[N- maleimidomethyl]cyclohexane-1-carboxy-[6-amidocaproate], N-[k-maleimidoundecanoyloxy]sulfosuccinimide ester, N-succinimidyl 3-(2-pyridyldithio)-propionate, succinimidyl 6-(3-[2-pyridyldithio]-propionamido)hexanoate, 4-succinimidyloxycarbonyl-methyl-a-[2-pyridyldithio]toluene, 4-sulfosuccinimidyl-6-methyl-a-[2-pyridyldithio)toluamido]hexanoate), N-succinimidyl iodoacetate, succinimidyl 3-[bromoacetamido]propionate, N-succinimidyl[4-iodoacetyl]aminobenzoate, N-sulfosuccinimidyl[4-iodoacetyl]aminobenzoate, N-hydroxysuccinimidyl-4-azidosalicylic acid, N-5-azido-2-nitrobenzoyloxysuccinimide, N-hydroxysulfosuccinimidyl-4-azidobenzoate, sulfosuccinimidyl[4-azidosalicylamido]-hexanoate, N-succinimidyl-6-(4′-azido-2′-nitrophenylamino) hexanoate, N-sulfosuccinimidyl-6-(4′-azido-2′-nitrophenylamino) hexanoate, sulfosuccinimidyl-(perfluoroazidobenzamido)-ethyl-1,3′-dithioproprionate, sulfosuccinimidyl-2-(m-azido-o-nitrobenzamido)-ethyl-1,3′-proprionate, sulfosuccinimidyl 2-[7-amino-4-methylcoumarin-3-acetamido]ethyl-1,3′-dithiopropionate, succinimidyl 4,4′-azipentanoate, succinimidyl 6-(4,4′-azipentanamido)hexanoate, succinimidyl 2-([4,4′-azipentanamido]ethyl)-1,3′-dithioproprionate, sulfosuccinimidyl 4,4′-azipentanoate, sulfosuccinimidyl 6-(4,4′-azipentanamido)hexanoate, sulfosuccinimidyl 2-([4,4′-azipentanamido]ethyl)-1,3′-dithioproprionate, dicyclohexylcarbodiimide, 1-ethyl-3-[3-dimethylaminopropyl]carbodiimide hydrochloride, N-[4-(p-azidosalicylamido) butyl]-3″-(2″-pyridyldithio)propionamide, N-[β-maleimidopropionic acid] hydrazide, trifluoroacetic acid salt, [N-e-maleimidocaproic acid] hydrazide, trifluoroacetic acid salt, 4-(4-N-Maleimidophenyl)butyric acid hydrazide hydrochloride, N-[k-maleimidoundecanoic acid]hydrazide, 3-(2-Pyridyldithio)propionyl hydrazide, p-azidobenzoyl hydrazide, N-[p-maleimidophenyl]isocyanate, and succinimidyl-[4-(psoralen-8-yloxy)]-butyrate.

DSC and SAC crosslinking of crosslinkable polynucleotides has been generally discussed above. Steps of the methods for these crosslinking strategies will largely mirror those recited above for polyalkyne crosslinking, except the activation step will not be optional for these crosslinking strategies. As described herein, a chemical is used to facilitate the crosslinking of crosslinkable polynucleotides. Thus, a nanosurface preparation step, a contacting step, activation step, incubation step, isolation step and optional dissolution step are carried out.

The above methods also optionally include a step wherein the structures further comprise an additional agent as defined herein. The additional agent can, in various aspects be added to the mixture during crosslinking of the polynucleotides, or can be added after production of the structure.

Binding Polynucleotides

The binding polynucleotide is a nucleic acid sequence that is sufficiently complementary to a polynucleotide target such that hybridization of the binding polynucleotide and the polynucleotide target takes place under physiological conditions.

As used herein, “physiological conditions” mean at least a temperature range of about 20 to about 40° C., atmospheric pressure of about 1, and pH of about 6 to about 8. In some instances, physiological conditions additional include a glucose concentration of about 1 to about 20 mM and atmospheric oxygen concentration.

The signal polynucleotide is a nucleic acid sequence that is sufficiently complementary to the binding polynucleotide such that hybridization of the binding polynucleotide and the signal polynucleotide takes place in the absence of target polynucleotide.

In various aspects, the binding polynucleotide is 100% complementary to the target polynucleotide, i.e., a perfect match, while in other aspects, the polynucleotide is at least (meaning greater than or equal to) about 95% complementary to the polynucleotide over the length of the polynucleotide, at least about 90%, at least about 85% complementary to the polynucleotide over the length of the polynucleotide to the extent that the polynucleotide is able to achieve the desired of inhibition of a target gene product. It will be understood by those of skill in the art that the degree of hybridization is less significant than a resulting detection of the target polynucleotide, or a degree of inhibition of gene product expression.

It is understood in the art that the sequence of the binding polynucleotide need not be 100% complementary to that of its target polynucleotide in order to specifically hybridize to the target polynucleotide. Moreover, a binding polynucleotide may hybridize to a target polynucleotide over one or more segments such that intervening or adjacent segments are not involved in the hybridization event (for example and without limitation, a loop structure or hairpin structure). The percent complementarity is determined over the length of the polynucleotide that is part of the structure. For example, given a structure comprising a polynucleotide in which 18 of 20 nucleotides of the polynucleotide are complementary to a 20 nucleotide region in a target polynucleotide of 100 nucleotides total length, the polynucleotide that is part of the structure would be 90 percent complementary. In this example, the remaining noncomplementary nucleotides may be clustered or interspersed with complementary nucleotides and need not be contiguous to each other or to complementary nucleotides. Percent complementarity of a polynucleotide that is part of a structure with a region of a target polynucleotide can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7:649-656).

The binding polynucleotide can be single stranded or double stranded. In some embodiments, the binding polynucleotide is single stranded. When it is double stranded, it may also includes a single strand sequence that hybridizes to complementary single stranded sequence of the target polynucleotide. Alternatively, hybridization of a binding polynucleotide can form a triplex structure with a target polynucleotide.

Binding polynucleotides useful in the present invention are those which modulate expression of a target polynucleotide. For example, antisense polynucleotides which hybridize to a target polynucleotide and inhibit translation, siRNA polynucleotides which hybridize to a target polynucleotide and initiate an RNAse activity (for example RNAse H), triple helix forming polynucleotides which hybridize to double-stranded polynucleotides and inhibit transcription, and ribozymes which hybridize to a target polynucleotide and inhibit translation can be used.

Each binding polynucleotides can be complementary to the same target polynucleotide. For example, if a specific mRNA is targeted, a single structure has the ability to bind to multiple copies of the same transcript. In one aspect, the structure comprises identical binding polynucleotides, i.e., each binding polynucleotide has the same length and the same sequence. In another aspect, the structure comprises binding polynucleotides which bind the same single target polynucleotide but at different locations, i.e., each may have differing lengths and/or sequences.

Alternatively two or more binding polynucleotides can be complementary to different target polynucleotides. In this embodiment, the binding polynucleotides can bind to different target polynucleotides which encode different gene products. Accordingly, in various aspects, a single structure may be used in a method to inhibit expression of more than one gene product. Binding polynucleotides are thus used to target specific polynucleotides, whether at one or more specific regions in the target polynucleotide or over the entire length of the target polynucleotide as the need may be to effect a desired level of inhibition of gene expression.

The binding polynucleotides are designed to complement the target sequence.

Alternatively, polynucleotides are selected from a library. Preparation of libraries of this type is well known in the art. See, for example, U.S. Published Application 2005/0214782.

Binding polynucleotides can also be aptamers. The production and use of aptamers is known to those of ordinary skill in the art. In general, aptamers are nucleic acid or peptide binding species capable of tightly binding to and discreetly distinguishing target ligands (Yan et al., RNA Biol. 6(3) 316-320 (2009), incorporated by reference herein in its entirety). Aptamers, in some embodiments, may be obtained by a technique called the systematic evolution of ligands by exponential enrichment (SELEX) process (Tuerk et al., Science 249:505-10 (1990), U.S. Pat. Nos. 5,270,163 and 5,637,459, each of which is incorporated herein by reference in their entirety). General discussions of nucleic acid aptamers are found in, for example and without limitation, Nucleic Acid and Peptide Aptamers: Methods and Protocols (Edited by Mayer, Humana Press, 2009) and Crawford et al., Briefings in Functional Genomics and Proteomics 2(1): 72-79 (2003). Additional discussion of aptamers, including but not limited to selection of RNA aptamers, selection of DNA aptamers, selection of aptamers capable of covalently linking to a target protein, use of modified aptamer libraries, and the use of aptamers as a diagnostic agent and a therapeutic agent is provided in Kopylov et al., Molecular Biology 34(6): 940-954 (2000) translated from Molekulyarnaya Biologiya, Vol. 34, No. 6, 2000, pp. 1097-1113, which is incorporated herein by reference in its entirety. In various aspects, an aptamer is between 10-100 nucleotides in length.

The binding polynucleotides can have the same or different sequences. When different polynucleotide sequences are present, each can hybridize to a different region on the same target polynucleotide. Alternatively, the different polynucleotide sequences hybridize to different polynucleotides, thereby modulating gene expression from different target polynucleotides.

Target Polynucleotides

Target polynucleotides bind to binding polynucleotides in the structure under physiological conditions. The structures described herein are used to bind to a target, which is a eukaryotic, prokaryotic, or viral polynucleotide.

In one embodiment, target polynucleotide is a mRNA encoding a gene product and translation of the gene product is inhibited upon binding to the structure. In another embodiment, the target molecule is a microRNA (miRNA). In another embodiment, the target polynucleotide is DNA in a gene encoding a gene product and transcription of the gene product is inhibited by the structure. In other embodiments, the target DNA is complementary to a coding region for the gene product. In still other aspects, the target DNA encodes a regulatory element necessary for expression of the gene product. “Regulatory elements” include, but are not limited to enhancers, promoters, silencers, polyadenylation signals, regulatory protein binding elements, regulatory introns, ribosome entry sites, and the like. In another embodiment, the target DNA is a sequence which is required for endogenous replication.

For prokaryotic target polynucleotides, the polynucleotide is genomic DNA or RNA transcribed from genomic DNA. For eukaryotic target polynucleotides, the polynucleotide is an animal polynucleotide, a plant polynucleotide, or a fungal polynucleotide, including yeast polynucleotides. As above, the target polynucleotide is either a genomic DNA or RNA transcribed from a genomic DNA sequence. In certain aspects, the target polynucleotide is a mitochondrial polynucleotide. For viral target polynucleotides, the polynucleotide is viral genomic RNA, viral genomic DNA, or RNA transcribed from viral genomic DNA.

It will be understood that one of skill in the art may readily determine appropriate targets and design and synthesize polynucleotides using techniques known in the art. Targets can be identified by obtaining, e.g., the sequence of a target nucleic acid of interest (e.g. from GenBank) and aligning it with other nucleic acid sequences using, for example, the MacVector 6.0 program, a ClustalW algorithm, the BLOSUM 30 matrix, and default parameters, which include an open gap penalty of 10 and an extended gap penalty of 5.0 for nucleic acid alignments.

Any essential prokaryotic gene is contemplated as a target polynucleotide. Exemplary genes include but are not limited to those required for cell division, cell cycle proteins, or genes required for lipid biosynthesis or nucleic acid replication. An essential prokaryotic gene for any prokaryotic species can be determined using a variety of methods including those described by Gerdes for E. coli [Gerdes et al., J Bacteriol. 185(19): 5673-84, 2003]. Many essential genes are conserved across the bacterial kingdom thereby providing additional guidance in target selection. Target polynucleotide sequences can be identified using readily available bioinformatics resources such as those maintained by the National Center for Biotechnology Information (NCBI).

For each of these three proteins, Table 1 of U.S. Patent Application Number 20080194463, incorporated by reference herein in its entirety, provides exemplary bacterial sequences which contain a target polynucleotide sequence for each of a number of important pathogenic bacteria. The gene sequences are derived from the GenBank Reference full genome sequence for each bacterial strain.

In another embodiment, binding polynucleotides of the structure hybridize to a sequence encoding a bacterial 16S rRNA nucleic acid sequence under physiological conditions, with a Tₘ substantially greater than 37° C., e.g., at least 45° C. and preferably 60° C.-80° C. Exemplary bacteria and associated GenBank Accession Nos. for 16S rRNA sequences are known in the art, and are provided, for example, in Table 1 of U.S. Pat. No. 6,677,153, incorporated by reference herein in its entirety.

In various embodiments, more than one target polynucleotide is detected in the target cell.

Signal Polynucleotides

The signal polynucleotide is a nucleic acid sequence that is sufficiently complementary to a binding polynucleotide such that hybridization between the binding polynucleotide and the signal polynucleotide occurs in the absence of target polynucleotide. Signal polynucleotides of the present invention have a lower binding affinity or binding avidity for the binding polynucleotide than the target such that association of the target molecule with the binding polynucleotide displaces and/or releases the signal polynucleotide from its association with the binding polynucleotide. When the signaling polynucleotide is not longer hybridized to the binding polynucleotide, the signal generated by the signal group detectably changes.

The signal polynucleotide can be single stranded or double stranded. In some embodiments, the signal polynucleotide is single stranded.

When it is double stranded, it may also include a single strand sequence that hybridizes to complementary single stranded sequence of the binding polynucleotide. Alternatively, hybridization of a signal polynucleotide can form a triplex structure with a binding polynucleotide. When the binding polynucleotide is DNA, the signaling polynucleotide is preferably a sicPN.

Each signal polynucleotide in the structure can be complementary to the same or different binding polynucleotides. In one embodiment, two or more signal polynucleotides bind to the same binding polynucleotide sequence. This embodiment allows binding of the structure to the target to be potentially monitored with two different signals.

Alternatively two or more signal polynucleotides can be complementary to different binding polynucleotides. In this embodiment, when different binding polynucleotides are present in the structure, the signal can be used to determine with binding polynucleotide bound to target.

Each signaling polynucleotide has one member of a signal/quencher pair attached, preferably covalently attached.

Suitable signal groups include those with a signal that is detectably changed when it is bound to the structure of the present invention. Suitable signal groups include fluorescent molecules, quantum dots, phosphorescent molecules, redox active probes, chemiluminescent molecules, radioactive labels, dyes, imaging and/or contrast agents, as well as any marker which can be detected using spectroscopic means, i.e., those markers detectable using microscopy and cytometry.

Preferably, the signal group is a fluorescent molecule whose signal is changed when it is in the proximity of a molecular quencher. Suitable fluorescent molecules useful as signal groups include without limitation 1,8-ANS (1-Anilinonaphthalene-8-sulfonic acid), 1-Anilinonaphthalene-8-sulfonic acid (1,8-ANS), 5-(and-6)-Carboxy-2′, 7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), BO-PRO-1-DNA, BO-PRO-3-DNA, BOBO-1-DNA, BOBO-3-DNA, BODIPY 650/665-X, MeOH, BODIPY FL conjugate, BODIPY FL, MeOH, Bodipy R6G SE, BODIPY R6G, MeOH, BODIPY TMR-X antibody conjugate pH 7.2, Bodipy TMR-X conjugate, BODIPY TMR-X, MeOH, BODIPY TMR-X, SE, BODIPY TR-X phallacidin pH 7.0, BODIPY TR-X, MeOH, BODIPY TR-X, SE, BOPRO-1, BOPRO-3, Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, Cl-NERF pH 6.0, Citrine, Coumarin, Cy 2, Cy 3, Cy 3.5, Cy 5, Cy 5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, Dil, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, Ethidium Bromide, Ethidium homodimer, Ethidium homodimer-1-DNA, eYFP (Enhanced Yellow Fluorescent Protein), FDA, FITC, FITC antibody conjugate pH 8.0, FlAsH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, high Ca, Fura-2, no Ca, GFP (S65T), HcRed, Hoechst 33258, Hoechst 33258-DNA, Hoechst 33342, Indo-1 Ca2+, Indo-1, Ca free, Indo-1, Ca saturated, JC-1, JC-1 pH 8.2, Lissamine rhodamine, LOLO-1-DNA, Lucifer Yellow, CH, LysoSensor Blue, LysoSensor Blue pH 5.0, LysoSensor Green, LysoSensor Green pH 5.0, LysoSensor Yellow pH 3.0, LysoSensor Yellow pH 9.0, LysoTracker Blue, LysoTracker Green, LysoTracker Red, Magnesium Green, Magnesium Green Mg2+, Magnesium Orange, Marina Blue, mBanana, mCherry, mHoneydew, MitoTracker Green, MitoTracker Green FM, MeOH, MitoTracker Orange, MitoTracker Orange, MeOH, MitoTracker Red, MitoTracker Red, MeOH, mOrange, mPlum, mRFP, mStrawberry, mTangerine, NBD-X, NBD-X, MeOH, NeuroTrace 500/525, green fluorescent Nissl stain-RNA, Nile Blue, EtOH, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, PicoGreen dsDNA quantitation reagent, PO-PRO-1, PO-PRO-1-DNA, PO-PRO-3, PO-PRO-3-DNA, POPO-1, POPO-1-DNA, POPO-3, Propidium Iodide, Propidium Iodide-DNA, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodaminen Green pH 7.0, Rhodol Green antibody conjugate pH 8.0, Sapphire, SBFI-Na+, Sodium Green Na+, Sulforhodamine 101, EtOH, SYBR Green I, SYPRO Ruby, SYTO 13-DNA, SYTO 45-DNA, SYTOX Blue-DNA, Tetramethylrhodamine antibody conjugate pH 8.0, Tetramethylrhodamine dextran pH 7.0, Texas Red-X antibody conjugate pH 7.2, TO-PRO-1-DNA, TO-PRO-3-DNA, TOTO-1-DNA, TOTO-3-DNA, TRITC, X-Rhod-1 Ca2+, YO-PRO-1-DNA, YO-PRO-3-DNA, YOYO-1-DNA, and YOYO-3-DNA.

Alternatively, the fluorescent molecule can be a fluorescent protein, preferably those selected from the list of proteins in the table below.

TABLE 1
Fluorescent Polypeptides

| | |
|---|---|
| EGFP | Emerald |
| CoralHue ® Azami Green | CoralHue ® Monomeric Azami |
| | Green |
| CopGFP | AceGFP |
| ZsGreen1 | TagGFP |
| TurboGFP | mUKG |
| | Blue/UV Proteins |
| EBFP | TagBFP |
| Azurite | EBFP2 |
| mKalama1 | GFPuv |
| Sapphire | T-Sapphire |
| | Cyan Proteins |
| ECFP | Cerulean |
| AmCyan1 | CoralHue ® Midoriishi-Cyan |
| TagCFP | mTFP1 |
| | Yellow Proteins |
| EYFP | Citrine |
| Venus | PhiYFP |
| TagYFP | TurboYFP |
| ZsYellow1 | |
| Orange Proteins | CoralHue ® Kusabira-Orange |
| CoralHue ® Monomeric Kusabira- | mKO_{K} |
| Orange | |
| mOrange | |
| Red Proteins | tdimer2(12) |
| mRFP1 | DsRed-Express |
| DsRed2 | DsRed-Monomer |
| HcRed1 | AsRed2 |
| eqFP611 | mRaspberry |
| mCherry | mStrawberry |
| mTangerine | tdTomato |
| TagRFP | JRed |
| TurboFP602 | |
| Far Red Proteins | mPlum |
| TurboFP635 | TagFP635 |
| AQ143 | HcRed-Tandem |
| | Large Stokes Shift Proteins |
| CoralHue ® mKeima Red | CoralHue ® dKeima Red |
| CoralHue ® dKeima570 | |
| Photoconvertible Proteins | CoralHue ® Kaede (green) |
| CoralHue ® Kaede (red) | CoralHue ® KikGR1 (green) |
| CoralHue ® KikGR1 (red) | KFP-Red |
| PA-GFP | PS-CFP |
| PS-CFP | mEosFP |
| mEosFP | CoralHue ® Dronpa |

Alternatively, the signaling group is a quantum dot. Quantum dots are semiconductor nanocrystals about 1 to about 20 nm in size. In comparison with organic dyes and fluorescent proteins, quantum dots represent a new class of fluorescent labels with unique advantages. For example, the fluorescence emission spectra of quantum dots can be continuously tuned by changing the particle size, and a single wavelength (typically in the blue or UV spectrum) can be used for simultaneous excitation of all different-sized quantum dots. Surface-passivated quantum dots are highly stable against photobleaching and have narrow, symmetric emission peaks (25-30 nm wide at half maximum intensity). Quantum dots also have high quantum yield: it has been estimated that CdSe quantum dots are about 20 times brighter and 100 times more stable than single rhodamine-6G molecules

The quantum dots of the present disclosure include a number of types of quantum dots such as, but not limited to, semiconductor, metal, and metal oxide nanoparticles (e.g., gold, silver, copper, titanium, nickel, platinum, palladium, oxides thereof (e.g., Cr₂O₃, CO₃O₄, NiO, MnO, CoFe₂O₄, and MnFeO₄), and alloys thereof), metalloid and metalloid oxide nanoparticles, the lanthanide series metal nanoparticles, and combinations thereof. In particular, semiconductor quantum dots are described in more detail below and in U.S. Pat. No. 6,468,808 and International Patent Application WO 03/003015, which are incorporated herein by reference.

Suitable quantum dots include, but are not limited to, luminescent semiconductor quantum dots. In general, quantum dots include a core and a cap, however, uncapped quantum dots can be used as well. The “core” is a nanometer-sized semiconductor. While any core of the IIA-VIA, IIIA-VA or IVA-IVA, IVA-VIA semiconductors can be used in the context of the present disclosure, the core must be such that, upon combination with a cap, a luminescent quantum dot results. A IIA-VIA semiconductor is a compound that contains at least one element from Group IIB and at least one element from Group VIA of the periodic table, and so on. The core can include two or more elements. In one embodiment, the core is a IIA-VIA, IIIA-VA or IVA-IVA semiconductor that ranges in size from about 1 nm to about 20 nm. In another embodiment, the core is more preferably a IIA-VIA semiconductor and ranges in size from about 2 nm to about 10 nm. For example, the core can be CdS, CdSe, CdTe, ZnSe, ZnS, PbS, PbSe or an alloy.

The “cap” is a semiconductor that differs from the semiconductor of the core and binds to the core, thereby forming a surface layer on the core. The cap can be such that, upon combination with a given semiconductor core a luminescent quantum dot results. The cap should passivate the core by having a higher band gap than the core. In one embodiment, the cap is a IIA-VIA semiconductor of high band gap. For example, the cap can be ZnS or CdS. Combinations of the core and cap can include, but are not limited to, the cap is ZnS when the core is CdSe or CdS, and the cap is CdS when the core is CdSe. Other exemplary quantum does include, but are not limited to, CdS, ZnSe, CdSe, CdTe, CdSeₓTe₁₋ₓ, InAs, InP, PbTe, PbSe, PbS, HgS, HgSe, HgTe, CdHgTe, and GaAs.

The wavelength emitted (i.e., color) by the quantum dots can be selected according to the physical properties of the quantum dots, such as the size and the material of the nanocrystal. Quantum dots are known to emit light from about 300 nanometers (nm) to 1700 nm (e.g., UV, near IR, and IR). The colors of the quantum dots include, but are not limited to, red, blue, green, and combinations thereof. The color or the fluorescence emission wavelength can be tuned continuously. The wavelength band of light emitted by the quantum dot is determined by either the size of the core or the size of the core and cap, depending on the materials which make up the core and cap. The emission wavelength band can be tuned by varying the structure and the size of the QD and/or adding one or more caps around the core in the form of concentric shells.

The intensity of the color of the quantum dots can be controlled. For each color, the use of 10 intensity levels (0, 1, 2, . . . 9) gives 9 unique codes (10¹⁻¹), because level “0” cannot be differentiated from the background. The number of codes increase exponentially for each intensity and each color used. For example, a three color and 10 intensity scheme yields 999 (10³⁻¹) codes, while a six color and 10 intensity scheme has a theoretical coding capacity of about 1 million (10″ In general, n intensity levels with m colors generate (nm^{m-1}) unique codes. Use of the intensity of the quantum dots has applications in quantum dots including a plurality of different types of quantum dots having different intensity levels and also in quantum dots including a plurality of different types of quantum dots having different intensity levels that are included in a porous material. The quantum dots are capable of absorbing energy from, for example, an electromagnetic radiation source (of either broad or narrow bandwidth), and are capable of emitting detectable electromagnetic radiation at a narrow wavelength band when excited. The quantum dots can emit radiation within a narrow wavelength band (FWHM, full width at half maximum) of about 40 nm or less, thus permitting the simultaneous use of a plurality of differently colored quantum dots with little or no spectral overlap.

The synthesis of quantum dots is well known and is described in U.S. Pat. Nos. 5,906,670; 5,888,885; 5,229,320; 5,482,890; 6,468,808; 6,306,736; 6,225,198, etc., International Patent Application WO 03/003015, (all of which are incorporated herein by reference) and in many research articles. The wavelengths emitted by quantum dots and other physical and chemical characteristics have been described in U.S. Pat. No. 6,468,808 and International Patent Application WO 03/003015 and will not be described in any further detail. In addition, methods of preparation of quantum dots are described in U.S. Pat. No. 6,468,808 and International Patent Application WO 03/003015 and will not be described any further detail.

Methods of attaching groups to polynucleotides are well known in the art. For example, conjugation of a contrast agent to a structure through a conjugation site is generally described in PCT/US2010/44844, which is incorporated herein by reference in its entirety.

Methods for visualizing the detectable change resulting from a fluorescent signal, include without limitation fluorescence microscopy, a microtiter plate reader or fluorescence-activated cell sorting (FACS). Methods for monitoring the detectable change resulting from a quantum dot signal, include without limitation.

“Quencher” as used herein, is a moiety that detectably changes the signal depending on whether it is close proximity or not. The signal need not be eliminated; rather it can be decreased or increased in a measurably way by the quencher.

When the signal is attached to the signaling moiety, the quencher is attached to the structure, preferably to either the crosslinkable polynucleotides or a spacer. When the signal is attached to the structure, the quencher is attached to the signaling moiety, preferably to either the crosslinkable polynucleotides or a spacer. The quencher is preferably covalently attached. The amount of quencher needed, depends on the amount of signaling moiety associated with the structure. The amount should be at least equal to, preferably at least twice as great, more preferably 10 times as great, as the signal group.

When a fluorescent molecule is used as a signal group, then the quencher includes, but is not limited to, dabcyl, malachite green, QSY 7, QSY 9m QSY 21, QSY 35, Iowa Black, Black Hole Quenchers, protein and peptides.

When a quantum dot is used the quencher can be any fluorophore to which energy can be transferred.

Additional Agents

The structures optionally can include one or more additional agents either entrapped in the interior of a hollow structure, covalently attached to a polynucleotide or spacer, enmeshed in the structural crosslinkable polynucleotides, or associated with one or both surfaces of the structure. Additional agents can be covalently or non-covalently associated with the structure.

Exemplary additional agents include biomolecules, coatings, polymeric agents, contrast agents, embolic agents, transcriptional regulators, therapeutic agents, and targeting moieties.

The structure of the present invention can comprise one or more additional agents. When structures of the present invention are used, each structure can contain one or more additional agents. Alternatively, at least two structures contain different additional agents. For example, a subset of structures could contain a therapeutic agent, while another subset contains a transcriptional regulator.

“Therapeutic agent,” “drug” or “active agent” as used herein means any compound useful for therapeutic purposes. The terms as used herein are understood to mean any compound that is administered to a patient (which can be animal or human) for the treatment of a condition.

Suitable therapeutic agents include both small molecules and biologics. Suitable therapeutic agents include, but are not limited to, the pharmaceutically active agents described in U.S. Pat. No. 7,611,728, which is incorporated by reference herein in its entirety. Other suitable therapeutic agents include, but are not limited to, the alkylating agents, antibiotic agents, antimetabolic agents, hormonal agents, plant-derived agents, and biologic agents described in U.S. Pat. No. 7,667,004 (incorporated by reference herein in its entirety).

In some aspects, the additional agent can be an antibiotic agents including, but not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

Suitable biologics useful as therapeutic agents include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM- CSF, granulocyte colony stimulating factor (G-CSF), interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, erythropoietin (EPO), thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including INFO, TNF1, TNF2, transforming growth factor a, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof. Examples of biologic agents include, but are not limited to, immuno-modulating proteins such as cytokines, monoclonal antibodies against tumor antigens, tumor suppressor genes, and cancer vaccines. Examples of interleukins that may be used in conjunction with the structures and methods of the present invention include, but are not limited to, interleukin 2 (IL-2), and interleukin 4 (IL-4), interleukin 12 (IL-12). Other immuno-modulating agents other than cytokines include, but are not limited to bacillus Calmette-Guerin, levamisole, and octreotide.

In one embodiment, methods and structures are provided wherein a therapeutic agent is able to traverse a cell membrane more efficiently when attached to a structure than when it is not attached to the structure. In various aspects, a therapeutic agent is able to traverse a cell membrane about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold or about 100-fold or more efficiently when attached to a structure than when it is not attached to the structure.

In various embodiments, the structure of the present invention serves as a drug delivery vehicle. As such, a significantly lower amount of therapeutic agent can be used compared to the amount needed in the absence of the structure of the present invention.

The term “targeting moiety” as used herein refers to any molecular structure which assists a compound or other molecule in binding or otherwise localizing to a particular target, a target area, entering target cell(s), or binding to a target receptor. Targeting moieties may include proteins, including antibodies and protein fragments capable of binding to a desired target site in vivo or in vitro, peptides, small molecules, anticancer agents, polynucleotide-binding agents, carbohydrates, ligands for cell surface receptors, aptamers, lipids (including cationic, neutral, and steroidal lipids, virosomes, and liposomes), antibodies, lectins, ligands, sugars, steroids, hormones, and nutrients, may serve as targeting moieties. Targeting moieties are useful for delivery of the structure to specific cell types and/or organs, as well as sub-cellular locations.

In some embodiments, the targeting moiety is a protein. Suitable targeting proteins may bind to a receptor, substrate, antigenic determinant, or other binding site on a target cell or other target site.

In some embodiments, the targeting moiety is an antibody. Suitable antibodies may be polyclonal or monoclonal. A number of monoclonal antibodies (MAbs) that bind to a specific type of cell have been developed. Antibodies derived through genetic engineering or protein engineering may be used as well. The antibody employed as a targeting agent in the present disclosure may be an intact molecule, a fragment thereof, or a functional equivalent thereof. Examples of antibody fragments useful in the structures of the present disclosure are F(ab′)2, Fab′ Fab and Fv fragments, which may be produced by conventional methods or by genetic or protein engineering.

In some embodiments, the crosslinkable polynucleotide of the structure may serve as an additional or auxiliary targeting moiety. The crosslinkable polynucleotide may be selected or designed to assist in extracellular targeting, or to act as an intracellular targeting moiety. That is, the polynucleotide portion may act as a DNA probe seeking out target cells. This additional targeting capability will serve to improve specificity in delivery of the structure to target cells.

The structures of the present invention can also include a transcriptional regulator. In these aspects, the transcriptional regulator induces transcription of a target polynucleotide in a target cell.

A transcriptional regulator as used herein is contemplated to be anything that induces a change in transcription of a mRNA. The change can, in various aspects, either be an increase or a decrease in transcription. In various embodiments, the transcriptional regulator is selected from the group consisting of a polypeptide, a polynucleotide, an artificial transcription factor (ATF) and any molecule known or suspected to regulate transcription.

In some embodiments, the transcription factor is a regulator polynucleotide. In certain aspects, the polynucleotide is RNA, and in further aspects the regulator polynucleotide is a noncoding RNA (ncRNA). In some embodiments, the noncoding RNA interacts with the general transcription machinery, thereby inhibiting transcription (Goodrich et al., Nature Reviews Mol Cell Biol 7: 612-616 (2006)). In general, RNAs that have such regulatory functions do not encode a protein and are therefore referred to as ncRNAs. Eukaryotic ncRNAs are transcribed from the genome by one of three nuclear, DNA-dependent RNA polymerases (Pol I, II or III). They then elicit their biological responses through one of three basic mechanisms: catalyzing biological reactions, binding to and modulating the activity of a protein, or base-pairing with a target nucleic acid. ncRNAs have been shown to participate actively in many of the diverse biological reactions that encompass gene expression, such as splicing, mRNA turn over, gene silencing and translation (Storz, et al., Annu. Rev. Biochem. 74: 199-217 (2005)). Notably, several studies have recently revealed that ncRNAs also actively regulate eukaryotic mRNA transcription, which is a key point for the control of gene expression.

In another embodiment, a regulatory polynucleotide is one that can associate with a transcription factor thereby titrating its amount. In some aspects, using increasing concentrations of the regulatory polynucleotide will occupy increasing amounts of the transcription factor, resulting in derepression of transcription of a mRNA.

In a further embodiment, a regulatory polynucleotide is an aptamer. The structures of the present invention can also include a coating. The coating can be any substance that is a degradable polymer, biomolecule or chemical that is non toxic. Alternatively, the coating can be a bioabsorbable coating. As used herein, “coating” refers to the components, in total, that are deposited on a structure. The coating includes all of the coated layers that are formed on the structure. A “coated layer” is formed by depositing a compound, and more typically a structure that includes one or more compounds suspended, dissolved, or dispersed, in a particular solution. As used herein, the term “biodegradable” or “degradable” is defined as the breaking down or the susceptibility of a material or component to break down or be broken into products, byproducts, components or subcomponents over time such as minutes, hours, days, weeks, months or years. As used herein, the term “bioabsorbable” is defined as the biologic elimination of any of the products of degradation by metabolism and/or excretion.

A non-limiting example of a coating that is a biodegradable and/or bioabsorbable material is a bulk erodible polymer (either a homopolymer, copolymer or blend of polymers) such as any one of the polyesters belonging to the poly(alpha-hydroxy acids) group. This includes aliphatic polyesters such poly (lactic acid); poly (glycolic acid); poly (caprolactone); poly (p-dioxanone) and poly (trimethylene carbonate); and their copolymers and blends. Other polymers useful as a bioabsorbable material include without limitation amino acid derived polymers, phosphorous containing polymers, and poly (ester amide). The rate of hydrolysis of the biodegradable and/or bioabsorbable material depends on the type of monomer used to prepare the bulk erodible polymer. For example, the absorption times (time to complete degradation or fully degrade) are estimated as follows: poly(caprolactone) and poly (trimethylene carbonate) takes more than 3 years; poly(lactic acid) takes about 2 years; poly(dioxanone) takes about 7 months; and poly (glycolic acid) takes about 3 months. Absorption rates for copolymers prepared from the monomers such as poly(lactic acid-co-glycolic acid); poly(glycolic acid-co-caprolactone); and poly(glycolic acid-co-trimethylene carbonate) depend on the molar amounts of the monomers.

The structures may also be administered by other controlled-release means or delivery devices that are well known to those of ordinary skill in the art. These include, for example and without limitation, hydropropylmethyl cellulose, other polymer matrices, gels, permeable membranes, multilayer coatings (see below), liposomes, or a combination of any of the above to provide the desired release profile in varying proportions. Other methods of controlled-release delivery of compounds will be known to the skilled artisan and are within the scope of the invention.

The therapeutic agent is, in some aspects, attached to a polynucleotide that is part of the structure, preferably it is attached to a subset of crosslinkable polynucleotides. Methods of attaching a therapeutic agent to a polynucleotide are known in the art, and are described in U.S. Pat. Nos. 5,391,723; 5,585,481;and 5,512,667 and U.S. Publication 2009/0209629, the disclosures of which are incorporated herein by reference in their entirety.

It will be appreciated that, in various aspects, a therapeutic agent as described herein is attached to the nanoparticle.

Methods of Using a Structure as Delivery Vehicles

Hollow structures are useful as a delivery vehicle. Thus, a hollow structure is made wherein an additional agent is localized inside the structure. In related aspects, the additional agent is associated with the structure as described herein. It is contemplated that the structure that is utilized as a delivery vehicle is, in some aspects, made more porous, so as to allow placement of the additional agent inside the structure. Porosity of the structure can be empirically determined depending on the particular application, and is within the skill in the art. All of the advantages of the functionalized nanoparticle (for example and without limitation, increased cellular uptake and resistance to nuclease degradation) are imparted on the hollow structure.

It is further contemplated that in some aspects the structure used as a delivery vehicle is produced with a polynucleotide that is at least partially degradable, such that once the structure is targeted to a location of interest, it dissolves or otherwise degrades in such a way as to release the additional agent. Biomolecule degradation pathways are known to those of skill in the art and can include, without limitation, nuclease pathways, protease pathways and ubiquitin pathways.

In some aspects, a structure of the disclosure acts as a sustained-release formulation. In these aspects, the structure is produced using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition [Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41, incorporated by reference herein in its entirety].

Methods of Detecting a Target Polynucleotide

The disclosure provides methods of detecting a target polynucleotide in a cell comprising contacting the cell with a structure as described. When the binding polynucleotide binds to the target polynucleotide, the signal is detectably changed. Detection of the signal can be by any of the methods described herein.

Prior to binding to target polynucleotide, the signaling moiety either is nondetectable (for example, when the signal group is quenched when in proximity with a quencher covalent attached to the structure. While it is understood in the art that the term “quench” or “quenching” is often associated with fluorescent markers, it is contemplated herein that the signal of any marker that is quenched when it is relatively undetectable. Thus, it is to be understood that methods exemplified throughout this description that employ fluorescent markers are provided only as single embodiments of the methods contemplated, and that any marker which can be quenched can be substituted for the exemplary fluorescent marker.

The signal moiety is thus associated with the structure in such a way that the detectable label is in proximity to the quencher. When the binding polynucleotide hybridizes with the target polynucleotide, the signal polynucleotide is displaced. The release of the signal polynucleotide thus increases the distance between the signal group present on the signaling polynucleotide and the surface to which the quencher is bound. This increase in distance allows detection of the previously quenched detectable label, and indicates the presence of the target polynucleotide. The amount of signal that is detected as a result of displacement of the signal polynucleotide is related to the amount of the target polynucleotide present in the cell. In general, an increase in the amount of signal correlates with an increase in the number of target polynucleotides present.

In some embodiments it is desirable to detect more than one target polynucleotide in a cell. In these embodiments, more than one signaling polynucleotide is used, and each comprises a different signal group with a unique signal. Accordingly, each target polynucleotide, as well as its relative amount, is individually detectable based on the detection of each signal.

Additional methods provided by the disclosure include methods of inhibiting expression of a gene product expressed from a target polynucleotide comprising contacting the target polynucleotide with a structure as described herein, wherein the contacting is sufficient to inhibit expression of the gene product.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of the inventive structure. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in vitro in a cell culture assay, generally as a predictable measure of a degree of inhibition that can be expected in vivo resulting from use of a structure as described herein. It is contemplated by the disclosure that the inhibition of a target polynucleotide is used to assess the effects of the inhibition on a given cell. By way of non-limiting examples, one can study the effect of the inhibition of a gene product wherein the gene product is part of a signal transduction pathway. Alternatively, one can study the inhibition of a gene product wherein the gene product is hypothesized to be involved in an apoptotic pathway.

It will be understood that any of the methods described herein can be used in combination to achieve a desired result. For example and without limitation, methods described herein can be combined to allow one to both detect a target polynucleotide as well as regulate its expression. In some embodiments, this combination can be used to quantitate the inhibition of target polynucleotide expression over time either in vitro or in vivo. The quantitation over time is achieved, in one aspect, by removing cells from a culture at specified time points and assessing the relative level of expression of a target polynucleotide at each time point. A decrease in the amount of target polynucleotide as assessed, in one aspect, through visualization of a detectable label, over time indicates the rate of inhibition of the target polynucleotide.

Local delivery of a structure to a human is contemplated in some aspects of the disclosure. Local delivery may optionally involve the use of an embolic agent in combination with interventional radiology and a composition of the disclosure.

Compositions

It will be appreciated that any of the structures described herein may be administered to a cell. In some embodiments, the structures are combined with an excipient to form a composition. The composition may contain two or more structures, which can be the same or different. When the composition is administered to a patient, the inventive structure is preferably combined with a pharmaceutically acceptable excipient.

When the composition is administered to a patient, the amount administered is a “therapeutically effective amount”, which means an amount of a structure sufficient to treat, ameliorate, or prevent the identified disease or condition, or to exhibit a detectable therapeutic, prophylactic, or inhibitory effect. The effect can be detected by, for example, an improvement in clinical condition, reduction in symptoms, or by an assay described herein. The precise effective amount for a subject will depend upon the subject's body weight, size, and health; the nature and extent of the condition; and the structure or combination of structures selected for administration. Therapeutically effective amounts for a given situation can be determined by routine experimentation that is within the skill and judgment of the clinician.

The structures described herein may be formulated in pharmaceutical structures with a pharmaceutically acceptable excipient, carrier, or diluent. The structure can be administered by any route that permits treatment of, for example and without limitation, a disease, disorder or infection as described herein. Additionally, the compound or structure comprising the composition may be delivered to a patient using any standard route of administration, including parenterally, such as intravenously, intraperitoneally, intrapulmonary, subcutaneously or intramuscularly, intrathecally, transdermally (as described herein), rectally, orally, nasally or by inhalation.

Slow release formulations may also be prepared from the agents described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or the compound of the embodiments can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition. Administration of combinations of therapeutic agents (i.e., combination therapy) is also contemplated, provided at least one of the therapeutic agents is in association with a structure as described herein.

In an embodiment, pharmaceutical compositions containing the structures and pharmaceutically acceptable excipients such as carriers, solvents, stabilizers, adjuvants, diluents, etc., are used. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions comprises in various aspects a therapeutically or prophylactically effective amount of at least one structure as described herein, together with one or more pharmaceutically acceptable excipients. As described herein, the pharmaceutical compositions may optionally comprise a combination of the compounds described herein.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular structure being administered, as well as by the particular method used to administer the structure. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using suitable dispersing or wetting agents and suspending agents. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

In some aspects of the disclosure, a method of dermal delivery of a structure is provided comprising the step of administering structure and a dermal vehicle to the skin of a patient in need thereof. The delivery of the structure can be transdermal or topical. In some embodiments, the dermal vehicle comprises an ointment. In some aspects, the ointment is Aquaphor.

In further embodiments of the methods, the administration of the structure ameliorates a skin disorder. In various embodiments, the skin disorder is selected from the group consisting of cancer, a genetic disorder, aging, inflammation, infection, and cosmetic disfigurement.

See PCT/US2010/27363, incorporated by reference herein in its entirety, for further description of dermal delivery of nanostructures and methods of their use.

In some embodiments, the structures of the present disclosure are suspended in a vehicles, including without limitation an ointment, cream, lotion, gel, foam, buffer solution (for example and without limitation, Ringer's solution and isotonic sodium chloride solution) or water. In some embodiments, vehicles comprise one or more additional substances including but not limited to salicylic acid, alpha-hydroxy acids, or urea that enhance the penetration through the stratum corneum.

In various aspects, vehicles contemplated for use in the structures of the present invention include, but are not limited to, Aquaphor® healing ointment, A+D, polyethylene glycol (PEG), glycerol, mineral oil, Vaseline Intensive Care cream (comprising mineral oil and glycerin), petroleum jelly, DML (comprising petrolatum, glycerin and PEG 20), DML (comprising petrolatum, glycerin and PEG 100), Eucerin moisturizing cream, Cetaphil (comprising petrolatum, glycerol and PEG 30), Cetaphil, CeraVe (comprising petrolatum and glycerin), CeraVe (comprising glycerin, EDTA and cholesterol), Jergens (comprising petrolatum, glycerin and mineral oil), and Nivea (comprising petrolatum, glycerin and mineral oil). One of ordinary skill in the art will understand from the above list that additional vehicles are useful in the structures and methods of the present disclosure.

An ointment, as used herein, is a formulation of water in oil. A cream as used herein is a formulation of oil in water. In general, a lotion has more water than a cream or an ointment; a gel comprises alcohol, and a foam is a substance that is formed by trapping gas bubbles in a liquid.

Administration of an embolic agent in combination with a structure of the invention is also contemplated. Embolic agents serve to increase localized drug concentration in target sites through selective occlusion of blood vessels by purposely introducing emboli, while decreasing drug washout by decreasing arterial inflow. Thus, the structure of the invention would remain at a target site for a longer period of time in combination with an embolic agent relative to the period of time the structure would remain at the target site without the embolic agent.

Suitable embolic agents to be used include without limiation a lipid emulsion (for example and without limitation, ethiodized oil or lipiodol), gelatin sponge, tris acetyl gelatin microspheres, embolization coils, ethanol, small molecule drugs, biodegradable microspheres, non-biodegradable microspheres or polymers, and self-assemblying embolic material.

The structures of the invention are administered by any route that permits imaging of the tissue or cell that is desired, and/or treatment of the disease or condition. In one aspect the route of administration is intraarterial administration. Additionally, the structure is delivered to a patient using any standard route of administration, including but not limited to orally, parenterally, such as intravenously, intraperitoneally, intrapulmonary, intracardiac, intraosseous infusion (“IO”), subcutaneously or intramuscularly, intrathecally, transdermally, intradermally, rectally, orally, nasally or by inhalation or transmucosal delivery. Direct injection of a structure provided herein is also contemplated and, in some aspects, is delivered via a hypodermic needle. Slow release formulations may also be prepared from the structures described herein in order to achieve a controlled release of the structure or a component of a structure so as to achieve substantially constant and effective levels in the blood plasma.

Methods provided also include those wherein a structure of the disclosure is locally delivered to a target site in a patient (non-human animal or human). Target cells for delivery of a structure of the disclosure are, in various aspects, selected from the group consisting of a cancer cell, a stem cell, a T-cell, and a β-islet cell. In one embodiment, the target site is a site of pathogenesis.

In some aspects, the site of pathogenesis is cancer. In various aspects, the cancer is selected from the group consisting of liver, pancreatic, stomach, colorectal, prostate, testicular, renal cell, breast, bladder, ureteral, brain, lung, connective tissue, hematological, cardiovascular, lymphatic, skin, bone, eye, nasopharyngeal, laryngeal, esophagus, oral membrane, tongue, thyroid, parotid, mediastinum, ovary, uterus, adnexal, small bowel, appendix, carcinoid, gall bladder, pituitary, cancer arising from metastatic spread, and cancer arising from endodermal, mesodermal or ectodermally-derived tissues.

In some embodiments, the site of pathogenesis is a solid organ disease. In various aspects, the solid organ is selected from the group consisting of heart, liver, pancreas, prostate, brain, eye, thyroid, pituitary, parotid, skin, spleen, stomach, esophagus, gall bladder, small bowel, bile duct, appendix, colon, rectum, breast, bladder, kidney, ureter, lung, and a endodermally-, ectodermally- or mesodermally-derived tissues.

Kits

The present invention also includes kits comprising at least one type of structure as disclosed. In one embodiment, the kit comprises at least one container, the container holding at least one type of structure as described. In another embodiment, the container holds two or more types of structures as described. In another embodiment, the kit comprises at least two containers. Each container holds a structure as disclosed herein different than the other. In another embodiment, the kit optionally includes one or more structures without binding polynucleotides (for use as controls).

### EXAMPLES

Experimental Details

All materials were purchased from Sigma-Aldrich Co., MO, USA, and used without further purification unless otherwise indicated. TEM characterization was conducted on a Hitachi H8100 electron microscope (Hitachi High-Tech Co., Japan). NMR experiments were performed using a Bruker Avance III 500 MHz coupled with a DCH CryoProbe (Bruker BioSpin Co., MA, USA). DLS data were acquired from a MALVERN Zetasizer, Nano-ZS (Malvern Instruments, UK). IR results were obtained from a Bruker TENSOR 37, and analyzed using the OPUS software (Bruker Optics Inc., MA, USA). MALDI-ToF measurements were carried out on a Bruker Autoflex III SmartBeam mass spectrometer (Burker Daltonics Inc., MA, USA). Fluorescence measurements were carried out with a Fluorolog-3 system (HORIBA Jobin Yvon Inc., NJ, USA). UV-Vis data are obtained on a Cary 5000 UV-Vis spectrophotometer (Varian Inc., CA, USA)

Oligonucleotide Synthesis

Oligonucleotides were synthesized on an Expedite 8909 Nucleotide Synthesis System (ABI) using standard solid-phase phosphoramidite methodology. Bases and reagents were purchased from Glen Research. Oligonucleotides were purified by reverse-phase high performance liquid chromatography (HPLC, Varian) on a Microsorb C18 column (Varian). To prepare the sequences used to make PNANs, strands were synthesized with a 3′ thiol group, 1-10 amino modifier-dT bases (5′-dimethoxytrityl-5[N- (trifluoroacetylaminohexyl)-3-acrylimido]-2′-deoxyuridine,3′-[(2-cyanoethyl)-(N,N- diisopropyl)]-phosphoramidite), 5 unmodified T bases, and ca. 20 standard bases for hybridization. After final removal of the dimethoxytrityl (DMT) protecting group and desalting, these strands were then reacted with an alkyne-NHS ester (3-propargyloxypropanoic acid N-hydroxysuccinimidyl ester, Quanta Biodesign) in an aqueous solution. In a typical reaction, 0.2 μmoles of the amine-modified oligonucleotides were dissolved in 500 μL of a 0.1 M carbonate/bicarbonate buffer (pH 9.0). To this solution was added 1 mg of the alkyne-NHS ester dissolved in 30 μL of DMSO. The reaction was allowed to proceed for 2 h and then desalted using an illustra NAP-10 column (GE Healthcare). The conversion was monitored using matrix-assisted laser desorption/ionization time of flight mass spectrometry (MALDI-TOF MS).

To synthesize strands for siRNA-PNANs, a sequence of 3′ thiol, 10 amino modifier-dT bases and 5 T bases were synthesized on an Expedite 8909 Nucleotide Synthesis System (ABI). The CPGs were then transferred to a column compatible for RNA synthesis and the RNA portion was continued as normal using TOM-RNA reagents (Glen Research) on a MerMade 6 (Bioautomation) RNA synthesizer. Strands were deprotected by standard methods and purified under RNAse free conditions. These strands were reacted with the alkyne-NHS ester as described above under RNAse free and otherwise identical conditions.

All sequences used for this study are as shown below (Table 1, “r” prior to the nucleobase indicates that the attached sugar is ribose; all other nucleobases are attached to deoxyribose)

| | | SEQ |
|---|---|---|
| | | ID |
| | | NO. |
| Size and Density | 5′ CCC AGC CTT CCA GCT CCT TG T₅-(T-alkyne)₁₀- | 1 |
| Analysis | SH 3′ | |
| NMR and IR | 5′ T₁₀-(T-alkyne)₁₀-SH 3′ | 2 |
| Analysis | | |
| PNAN EGFR | 5′ rCrArA-rArGrU-rGrUrG-rUrArA-rCrGrG-rArArU-rA- | 3 |
| Sense | T₅-(T-alkyne)₁₀-SH 3′ | |
| EGFR Sense | 5- rCrArA-rArGrU-rGrUrG-rUrArA-rCrGrG-rArArU-rA | 4 |
| | T₅-(T-alkyne)₁₀-SH | |
| EGFR Antisense | 5′ rUrArU-rUrCrC-rGrUrU-rArCrA-rCrArC-rUrUrU-rG 3′ | 5 |
| PNAN SCRAM | 5′rArUrC-rGrArA-rUrUrC-rCrUrG-rCrArG-rCrCrC- | 6 |
| Sense | rGrUrU-T₅-(T-alkyne)₁₀-SH 3′ | |
| SCRAM Sense | 5′rArUrC-rGrArA-rUrUrC-rCrUrG-rCrArG-rCrCrC- | 7 |
| | rGrUrU-SH 3′ | |
| SCRAM Antisense | 5′ rArArC-rGrGrG-rCrUrG-rCrArG-rGrArA-rUrUrC- | 8 |
| | rGrArU 3′ | |
| PNAN Dabcyl for | 5′CTT-GAG-AAA-GGG-C(Dabcyl-T)G-CCA-T₅-(T- | 9 |
| Nuclease | alkyne)₁₀-SH 3 | |
| Nuclease FITC | 5′ Fluorescein-TTG GCA GCC C -3′ | 10 |
| Reporter | | |
| PNAN Cell Imaging | 5′Cy5-CAG-CTG-CAC-GCT-GCC-CTC-T₅-(T-alkyne)₁₀- | 11 |
| | SH 3′ | |
| FITC PNAN Melt | 5′ TCA-CT_{FITC}A-TTA-T₅-(T-alkyne)₁₀-SH 3′ | 12 |
| Strand | | |
| Cy3 PNAN Melt | 5′ TAA-T_{Cy3}AG-TGA-T₅-(T-alkyne)₁₀-SH 3′ | 13 |
| Strand | | |

Gold Particles for Density and Size Analyses

For density and size analyses, 5, 10, 20, 30 nm citrate stabilized gold nanoparticles were purchased from Ted Pella.

Gold Nanoparticles for Nuclease Resistance and Cell Studies

For all nuclease resistance and cell studies, citrate-stabilized gold nanoparticles (13 nm) were prepared using published procedures (G. Frens, Nature Phys. Sci. 241, 20 (1973)). These particles were then rendered DNAse and RNAse free using a protocol recently developed in our group (D. A. Giljohann et al., J. Am. Chem. Soc. 131, 2072 (2009)). Briefly, as synthesized particles were treated with 0.1% diethylpyrocarbonate (DEPC) for 12 h with stirring, then autoclaved at 121° C. for 60 min. This preparation caused no differences in particle morphology as determined by UV spectroscopy or Transmission Electron Microscopy (TEM) analysis as previously reported. Subsequent ligand functionalization also was not affected by this treatment.

General Method to Synthesize DNA PNANs

In a typical synthesis, 30 nmol of purified PNAN alkyne DNA strands were treated with 50 μL of 100 mM dithiothreitol (DTT, Pierce Biotechnology) in 50 mM pH 8.0 phosphate buffer for 1 h and desalted using a G25 illustra NAP-10 column (GE Healthcare). These purified strands were then added to 10 mL of gold nanoparticles of 5-10 nM. After 30 min, 10 μL of 10% TWEEN-20 was added to the particle/DNA solution and was brought to 50° C. Over 6 hs, the particles were brought to the elevated salt concentration by adding aliquots of 5 M NaCl (e.g. 10 mL of particles salt-aged to 1 M NaCl required 10 250 μL additions of 5 M NaCl.). The particles were allowed to shake for 48 h at 50° C. Particles were then purified by centrifugation using speeds appropriate for the size of particle and resuspended in NanopureTM water. This process was repeated 3 times. On the final centrifugation, particles were resuspended into ca. 500 μL of NanopureTM water. To this solution was added 30 μL of 1M KCN and the mixture was allowed to shake on a thermomixer at 800 rpm (Eppendorf) for 30 min. Successful PNAN synthesis will result in gold particles dissolving without any aggregation. Residues were removed by extensive dialysis using dialysis membrane with a MWCO of 5 kDa against 0.5 M NaCl solution for 2 days, then against nanopure water for 1 day. For cellular studies, the final dialysis is performed in 1× PBS buffer.

General Method to Synthesize RNA PNANs

In a typical synthesis, 30 nmol of purified PNAN alkyne-RNA sense strands were treated with 50 μL of 100 mM DTT (Thermo Scientific, RNAse Free) in 50 mM autoclaved pH 8.0 phosphate buffer for 1 h and desalted using a G25 illustra NAP-10 column (GE Healthcare). These strands were added to 10 mL of autoclaved 13 nm AuNPs (vide infra). After 30 min 10 μL of RNAse free TWEEN 20 was added, and the solution was incubated at 50° C. Over 6 h, the particles were brought to 1 M NaCl by adding aliquots of 5 M NaCl (e.g. 10 mL of particles salt-aged to 1 M NaCl required ten 250 μL additions of 5 M NaCl). The particles were allowed to shake for 48 h at 50° C. Particles were then purified by centrifugation at 15 kRPM for 30 min and resuspended in 0.15 M PBS. To this solution was added 15 nmol of the appropriate antisense RNA strand, and the solution was then heated to 60° C. for 30 min. The particles were then allowed to cool to room temperature and shake for at least 2 h before centrifugation at 15 kRPM @ 4° C. and resuspension in RNAse free PBS buffer. This process was repeated 2 times and on the final step, particles were concentrated into ca. 500 μL.

Particles were loaded into a pre-soaked Slide-A-Lyzer dialysis cassette (0.1-0.5 mL volume, 5 kDa MWCO, Thermo Scientific) using an RNAse free syringe/needle. These siRNA PNANs were dialyzed against RNAse free 0.5 M NaCI solution for 2 days, and then against RNAse free PBS buffer for 1 day. Particles were then isolated with an RNAse free syringe/needle and kept at 4° C. until used (typically within 1 h).

32P Radiolabeling of PNANs

Alkyne-DNA (30 nmol) was dissolved in 56 uL of Nanopure™ water. To this solution was added 10 ul of Kinase 10× Buffer (Promega), 4 μL of T4 polynucleotide kinase (Promega), and 30 μl of [−32P] ATP (at 3,000 Ci/mmol, 10 mCi/ml, 50 pmol total, Perkin Elmer). This solution was allowed to shake at 37° C. for 30 min and then 50 μL of 300 mM DTT was added and allowed to shake for an additional 30 min. The solution was then desalted using an illustra NAP-10 column (GE Healthcare). These strands AuNPwere used to form PNANs following identical procedures used for synthesizing PNANs (vide infra).

Partial AuNP Dissolution for TEM and UV-Vis Analysis

Upon addition of KCN to alkyne-DNA coated AuNPs, small aliquots (50 μL) were extracted at different time points during the dissolution process, and were added to pre-washed Nanosep100 kDa MWCO spin filters (Pall Life Sciences) and rinsed 5 times following manufacturer's protocol. Isolated partially dissolved AuNPs were then used for UV-Vis and TEM analysis.

Gel Electrophoresis of PNANs

All gel experiments were done in a 1% agarose/ethidium bromide gel in 1× TBE (tris, borate, EDTA) buffer. A 500 by EZ-Load ladder (Biorad) was used for all experiments. Gels were run at 100 V for 30 min using a Biorad PowerPac. Images were taken using a Fluorochem Q (Cell Biosciences) with an EBR-500K ethidum bromide filter.

Quantification of Strands/Particle

Radiolabeled PNAN/AuNPs were measured against a standard curve of 32P radiolabeled alkyne DNA using a TriCarb 2910 TR Liquid Scintillation Counter (Perkin Elmer). In a typical measurement, 100 μL of DNA or labeled AuNPs at varying concentrations were added to 2 mL of Ultima Gold™ scintillation cocktail (Perkin Elmer). The alkyne-DNA- AuNPs were then measured using GeneQuant 1300 (GE Healthcare) UV-Vis to determine their concentration. The extinction coefficients used for the AuNPs are as follows: 5 nm: 9.696×106 M−1cm−1, 10 nm: 9.55×106 M−1cm−1, 20 nm: 9.406×106 M−1cm−1, 30 nm: 3.859×106 M−1cm−1.

Hybridization and Melting Assays

PNANs were prepared at final concentrations of ˜150 nM and combined in equal volumes (30 μL for each sample) in 0.5M NaCl. The aggregates formed instantly and were allowed to settle for ˜6 h. The aggregates were centrifuged at 500 rpm for 20 seconds and the supernatant was removed. The aggregates were washed 3 times using 500 μL of 0.5M NaCl. Finally, a small amount of aggregates were removed and added to 1.0 mL of 0.5 M NaCl with 0.01% TWEEN 20. The suspension was placed into a Cary 5000 UV-Vis spectrometer equipped with temperature control accessory. Temperature was ramped at 0.25° C./minute from 20° C. to 80° C. and absorbance was monitored at 260 nm for DNA and PNANs and at 520 nm for AuNP-DNA conjugates. Fluorescence spectra were taken at room temperature and at 80° C. Photographs were taken using a Canon xsi CCD camera while the samples were suspended above a Spectroline ENF-240C UV-lamp emitting at 365 nm.

Nuclease Degradation Kinetics

Fluorophore-labeled complementary DNA was hybridized (1000 nM) to AuNP-DNA conjugates or PNANs (100 nM) in PBS, resulting in 10 fluorophores for every 1 nanoparticle. For the dabcyl-labeled free-DNA system, both strands were 1000 nM. To anneal the DNA, the solutions were heated to 70° C. for 1 hour and allowed to cool slowly to room temperature (˜12 hours). Once hybridized the samples were diluted to 1 nM in assay buffer (10 mM tris (pH =7.5), 2.5 mM MgCl2, and 0.5 mM CaCl2) and DNase I (20 units/mL, New England BioLabs) was added. The change in fluorescence overtime was monitored in 96 well plate format using a BioTek, Synergy H4 Hybrid Reader. The fluorescence of the sample (excitation =490 nm, emission =530 nm) was measured every 30 seconds for 2 hours. All samples were measured at least 5 times.

Cell Culture

SCC12 cells were grown in Gibco® DMEM/F12 (Invitrogen), with 10% heat inactivated fetal bovine serum and maintained at 37° C. in 5% CO2.

Confocal Microscopy of PNANs

To visualize the cellular uptake of PNANs, SCC12 cells were grown on Lab-Tek®II Chamber #1.5 German Coverglass System (Nalge Nunc International) overnight and incubated with 1 nM Cy5-labled PNANs. After 24 hours of incubation, the media was replaced with fresh media and live cells were stained with Hoechst 33342 (Invitrogen) following the manufacturer's instructions. All images were obtained with a Zeiss 510 LSM at 40× magnification using a Mai Tai 3308 laser (Spectra-Physics). Fluorescence emission was collected at 390-465 nm and 650-710 nm, exciting at 729 and 633 nm respectively.

Cellular Uptake of PNANs

Cells were seeded in 48 well plates and were grown for 24 h to reach 40% confluency prior to treatment. The cells were incubated with radio-labeled PNANs or AuNPs (1, 5, 10 nM) for 24 h and cells without any treatment were used as blank control. After treatment, cells were washed 3 times in PBS buffer, trypsinized and counted using a Countess® Automated Cell Counter (Invitrogen). To prepare samples for radioactivity measurement, cells were spun down and dissolved with 1× cell lysis buffer (Cell Signaling). The radioactivity of cell lysate was measured as described above. The number of PNAN/AuNPs in each sample was calculated based on the concentration of radiolabeled DNAs. Once the number of PNANs/AuNPs was calculated, this number was divided by the cell count to determine the number of PNAN/AuNPs per cell. All experiments were performed in triplicate and averaged.

EGFR Protein Knockdown Experiments (mRNA quantification and Western blotting)

For all the knockdown experiments, SCC12 cells were plated in 6-well plates at the density of 5000 cell/mL and incubated overnight. Cells were incubated with anti-EGFR siRNA-PNANs, siRNA-AuNPs or siRNA that complexed with DharmaFECT® 1 (Dharmacon) following the manufacturer's recommended protocol. After 48 h, the medium was replaced with fresh one and incubated for another 12 h. Scrambled siRNA-PNAN-treated cells and untreated cells were used as controls.

To quantify EGFR knockdown at mRNA level, cells were harvested and total RNA was extracted using TRIzol reagent (Invitrogen) followed by treatment with DNase I (Invitrogen) according to the manufacturer's protocol. RNA (1 μg) was reverse transcribed using qScript cDNA SuperMix (Quanta BioSciences). Real-time reverse-transcription PCR was performed on cDNA with LightCylcer®480 SYBR Green I Master on a LightCycler®480 system (Roche). The relative abundance of each mRNA transcript was normalized to GAPDH expression and compared to untreated cells to determine the increased expression. The standard deviation for this data was calculated from three independent experiments. The primers for human genes used in this experiment were EGFR forward, 5′-GCC GCA AAG TGT GTA ACG GAA TAG-3′ (SEQ ID NO. 14), EGFR reverse, 5′-TGG ATC CAG AGG AGG AGT ATG TGT-3′ (SEQ ID NO. 15), GAPDH forward, 5′-TGC ACC ACC AAC TGT TTA GC-3′ (SEQ ID NO. 16), GAPDH reverse, 5′-GGC ATG GAC TGT GGT CAT GAG-3′ (SEQ ID NO. 17). The primers were obtained from Integrated DNA Technologies.

To analyze EGFR knockdown at protein level, cells were treated with anti-EGFR siRNA-PNANs, anti-EGFR siRNA-AuNPs or DharmaFECT 1-complexed anti-EGFR siRNAs as described above. The whole cell lysates were prepared in 100 μL of Cell Lysis Buffer with 1 mM PMSF (Cell Signaling Technology) according to the protocol suggested by manufacturer. Protein concentrations were determined using BCA Protein Assay Kit (Pierce). Equal amounts (10 μg) of protein samples were fractionated by 7.5% SDS-PAGE and transferred to Hybond ECL membrane and analyzed by western blotting with EGFR (sc-03) and GAPDH (sc-32233) antibodies (Santa Cruz) using ECL Western Blotting Substrate (Pierce).

Cytotoxicity

The cytotoxicity of PNANs was evaluated with Vybrant® MTT Cell Proliferation Kit (Molecular Probes). Briefly, SCC12 cells were seeded on a 96 well plate in 100 pL media and incubated for 24 h. The cells were then treated with PNANs at varying concentrations of total DNAs (0.1, 0.5, 1, 2.5, 5, and 10 μg). Lipofectamine 2000 (Invitrogen) was used as a comparison in this study to further assess cytotoxicity of PNANs. Cells were transfected with different amount of DNAs (0.25, 0.5, 1, 2.5 μg) with Lipofectamine 2000 under manufacturer's instructions. Cells without treatment were used as a negative control. After 24 h, medium was removed, cells were washed with PBS for 3 times and then incubated with 100 μL fresh culture medium with addition of 10 μL of freshly-made 12 mM MTT solution at 37° C. in 5% CO2 for 4 h. 100 μL lysis buffer (1 g of SDS in 10mM of 0.01 M HCl) per well was added. Cells were further incubated overnight and the absorbance was measured at 570 nm using a Multiskan® Spectrum (Thermo Scientific). Each condition was repeated in triplicates in three independent experiments.

1. A structure formed from crosslinked polynucleotides, comprising:
a plurality of crosslinkable polynucleotides that are crosslinked; where a subset of the crosslinkable polynucleotides are binding polynucleotides that are sufficiently complementary to a target to allow them to hybridize under physiological conditions;
a plurality of signaling moieties hybridized to at least some of the binding polynucleotides in the structure, where each signaling moiety comprise either a quencher or a signal group attached to a signal polynucleotide which is sufficiently complementary to the binding polynucleotide to allow it to hybridize under physiological conditions,
when the signaling moiety comprises the quencher, then the signal group is bound to the structure, or when the signaling moiety comprises the signal group, then the quencher is bound to the structure;
where lack of hybridization leads to a detectably change in the signal.

2. The structure of claim 1, which is metal free.

3. The structure of claim 1, which is hollow.

4. The structure of claim 1, further comprising a spacer, wherein the crosslinkable polynucleotides are crosslinked through the spacer.

5. The structure of claim 1, where the quencher is bound to the structure.

6. The structure of claim 1, where the signal is bound to the structure.

7. The structure of claim 1, wherein the crosslinkable polynucleotides are crosslinked via amine, amide, alcohol, ester, aldehyde, ketone, thiol, disulfide, carboxylic acid, phenol, imidazole, hydrazine, hydrazone, azide, or alkyne groups.

8. The structure of claim 7, wherein the crosslinkable polynucleotides are crosslinked via alkyne groups.

9. The structure of claim 1, wherein the crosslinkable polynucleotides are about 2 to about 100 nucleotide bases in length.

10. The structure of claim 1, where the signal polynucleotides are about 2 to about 100 nucleotide bases in length.

11. The structure of claim 1, where the signal group is a fluorescent, colorimetric, radioactive, chemiluminescent, NIR active, magnetic, catalytic, or enzymatic group or are quantum dots.

12. The structure of claim 1, where the signal group is a fluorescent group or a quantum dot.

13. The structure of claim 1, where at least some of the quenchers are covalently attached to the crosslinkable polynucleotides.

14. The structure of claim 10, where the quencher is dabcyl, malachite green, QSY 7, QSY 9, QSY 21 , QSY 35, Iowa Black and Black Hole Quenchers.

15. The structure of claim 1, where at least 5% of the crosslinked polynucleotides are binding polynucleotides.

16. The structure of claim 1, where at least 5% of the binding polynucleotides are hybridized to signal polynucleotides.

17. The structure of claim 1, further comprising an additional agent entrapped in the interior of the structure, covalently attached to structure, enmeshed in the crosslinked polynucleotides, or associated with a surface of the structure.

18. A composition comprising an excipient and the structure of claim 1.

19. The composition of claim 18, wherein the carrier is a pharmaceutically acceptable excipient.

20. The composition of claim 18, further comprising an additional agent entrapped in the interior of the structure, covalently attached to structure, enmeshed in the crosslinked polynucleotides, or associated with a surface of the structure.

21. A method of detecting the presence of a target polynucleotide in a cell in vitro, comprising the steps of:
(a) contacting a cell in solution with the structure of claim 1 for a time sufficient to allow the cell to internalize the structure,
(b) monitoring the cell for signal,
wherein a detectable increase in signal indicates the presence of target polynucleotide in the cell.

22. A method of detecting the presence of a target in a cell in vivo, comprising the steps of:
(a) contacting a cell in a patient with the structure of claim 1 for a time sufficient to allow the structure to internalize into at least one cell in the patient,
(b) monitoring the cell for signal,
wherein a detectable increase in signal indicates the presence of target in the cell.

23. The method of claim 22, wherein the patient in a non-human animal or a human.

24. The method of claim 22, wherein the contacting is by administering the structure of claim 1 to a patient parenterally, intraperitoneally, intrapulmonary, subcutaneously, intramuscularly, intrathecally, transdermally, rectally, orally, nasally or by inhalation.

25. The method of claim 22, wherein the structure of claim 1 is delivered to an organ or tissue in the patient.

26. The method of claim 21, where the target is coding DNA, non-coding DNA, or miRNA.

27. The structure of claim 1, where the quencher is covalently bound to the structure or the signaling polynucleotide.

28. The structure of claim 1, where the signal group is covalently bound to the structure or the signaling polynucleotide.


SEQUENCE LISTING (17 sequences)

SEQ ID NO: 1 - DNA, 35 bp, Artificial Sequence
  Synthetic Polynucleotide
cccagccttccagctccttgttttttttttttttt

SEQ ID NO: 2 - DNA, 20 bp, Artificial Sequence
  Synthetic Polynucleotide
tttttttttttttttttttt

SEQ ID NO: 3 - DNA, 34 bp, Artificial Sequence
  Synthetic PNAN EGFR Sense
caaaguguguaacggaauattttttttttttttt

SEQ ID NO: 4 - DNA, 34 bp, Artificial Sequence; identical to SEQ ID NO: 3
  Synthetic EGFR Sense

SEQ ID NO: 5 - RNA, 19 nt, Artificial Sequence
  Synthetic EGFR Antisense
uauuccguuacacacuuug

SEQ ID NO: 6 - DNA, 36 bp, Artificial Sequence
  Synthetic PNAN SCRAM Sense
aucgaauuccugcagcccguuttttttttttttttt

SEQ ID NO: 7 - RNA, 21 nt, Artificial Sequence
  Synthetic SCRAM Sense
aacgggcugcaggaauucgau

SEQ ID NO: 8 - RNA, 21 nt, Artificial Sequence
  Synthetic SCRAM Antisense
aacgggcugcaggaauucgau

SEQ ID NO: 9 - DNA, 33 bp, Artificial Sequence
  Synthetic Polynucleotide (PNAN Dabcyl for Nuclease)
cttgagaaagggctgccattttttttttttttt

SEQ ID NO: 10 - DNA, 10 bp, Artificial Sequence
  Synthetic Polynucleotide (Nuclease FITC Reporter)
ttggcagccc

SEQ ID NO: 11 - DNA, 33 bp, Artificial Sequence
  Synthetic PNAN for Cell Imaging
cagctgcacgctgccctcttttttttttttttt

SEQ ID NO: 12 - DNA, 24 bp, Artificial Sequence
  Synthetic FITC PNAN Melt Strand
tcactattattttttttttttttt

SEQ ID NO: 13 - DNA, 24 bp, Artificial Sequence
  Synthetic Cy3 PNAN Melt Strand
taatagtgattttttttttttttt

SEQ ID NO: 14 - DNA, 24 bp, Artificial Sequence
  Synthetic Polynucleotide
gccgcaaagtgtgtaacggaatag

SEQ ID NO: 15 - DNA, 24 bp, Artificial Sequence
  Synthetic Polynucleotide
tggatccagaggaggagtatgtgt

SEQ ID NO: 16 - DNA, 20 bp, Artificial Sequence
  Synthetic Polynucleotide
tgcaccaccaactgtttagc

SEQ ID NO: 17 - DNA, 21 bp, Artificial Sequence
  Synthetic Polynucleotide
ggcatggactgtggtcatgag